(19) United States
(12) Patent Application Publication
(10) Pub. No.: US 2019/0056323 A1
(43) Pub. Date: Feb. 21, 2019

(54) FLUORESCENCE DETECTION APPARATUS, ANALYSIS METHOD, AND FLUORESCENCE DETECTION SYSTEM

(71) Applicants: SHISEIDO COMPANY, LTD., Tokyo (JP); KYUSHU UNIVERSITY, NATIONAL UNIVERSITY CORPORATION, Fukuoka (JP)

(72) Inventors: Masashi MITA, Tokyo (JP); Eiichi NEGISHI, Kyoto (JP); Kazuhiko Mibayashi, Kyoto (JP); Kenji HAMASE, Fukuoka (JP)

(21) Appl. No.: 15/770,593

(22) PCT Filed: Nov. 4, 2016
(86) PCT No.: PCT/JP2016/082842

§ 371 Date: Apr. 24, 2018

(30) Foreign Application Priority Data
Nov. 6, 2015 (JP) PCT/JP2015/081389

(51) Int. Cl. G01N 21/64; G01N 30/74; G01N 33/50; G01N 30/46

(52) U.S. Cl. CPC G01N 21/645; G01N 30/74; G01N 2201/0627; G01N 30/463; G01N 2021/6419; G01N 33/50

(57) ABSTRACT

[Problem]

To provide a fluorescence detection apparatus that is capable of simultaneously detecting a plurality of analyte components contained at different concentrations with a wide measurement range.

[Solution]

A fluorescence detection apparatus 100 includes a cell 110 into which an analyte of a sample is introduced, a light source 130 that irradiates excitation light on the analyte in the cell; a first detector 52 that detects fluorescence generated from the analyte after the excitation light has been irradiated on the analyte, and a second detector 53 that detects the fluorescence generated from the analyte after the excitation light has been irradiated on the analyte. The second detector detects the fluorescence with a measurement range that is different from the measurement range of the first detector, and the first detector and second detector detect the fluorescence simultaneously.

### TECHNICAL FIELD

The present invention relates to a fluorescence detection apparatus, an analysis method using the fluorescence detection apparatus, and a fluorescence detection system including the fluorescence detection apparatus.

The present application is based on and claims the benefit of priority of PCT International Application No. PCT/JP2015/081389 filed on Nov. 6, 2015, the entire contents of which are herein incorporated by reference.

### BACKGROUND ART

Generally, concentration sensitive detectors, such as ultraviolet-visible spectroscopic detectors and fluorescence detectors, can indicate accurate quantitation only within a range (measurement range) in which linearity of the calibration curve can be achieved. In a case where analyte components in a sample are included at various concentrations over a wide concentration range beyond the measurement range of the detector, conventionally, a plurality of analyses with measurement ranges including the respective concentrations of the components have to be conducted, or a plurality of detection means having measurement ranges including the respective concentrations of the components have to be implemented, step by step, in a single analysis.

In this respect, a technique is known for detecting a plurality of components having different concentrations by connecting two cells in series and detecting each component using a different detector to thereby enhance the measurement range (Patent Document 1). Also, a technique is known that involves selecting one detector from among two types of detectors having different sensitivities as the detector for a given cell in detecting a component (Patent Document 2).

### PRIOR ART DOCUMENTS

### Patent Documents

### Patent Document 1: Japanese Unexamined Patent Publication No. 2007-212235

### Patent Document 2: Japanese Unexamined Patent Publication No. 2015-022021

### SUMMARY OF THE INVENTION

### Problem to be Solved by the Invention

In the above Patent Document 1, the measurement range can be enhanced by using two types of detectors having different sensitivities. However, by arranging cells in two stages, separation of the analyte component may be degraded as a result of increased chances of diffusion of the analyte component, and quantitation accuracy may be degraded as a result of irreversible decomposition of the analyte component within a cell in optical detection. Also, it has been difficult to set up optimized conditions for each of the two different detection means connected in series.

Also, in the above Patent Document 2, switching means for switching between two detectors is provided so that a desired measurement range for an analyte component can be selected. However, because this technique only enables use of the measurement range of one detector with respect to one fraction, a plurality of analyses may be required when the range is exceeded. This can be a fatal problem when analyzing a rare biological sample for which restrictions are imposed on the frequency of analysis. Also, a control mechanism having a complicated algorithm has been required to avoid such a problem.

In view of the above problems of the related art, one aspect of the present invention is directed to providing a fluorescence detection apparatus that can simultaneously measure a plurality of analyte components having different concentrations within a wide measurement range.

### Means for Solving the Problem

According to one embodiment of the present invention, a fluorescence detection apparatus is provided that includes a cell into which an analyte of a sample is introduced, a light source configured to irradiate excitation light on the analyte in the cell, a first detector configured to detect fluorescence generated from the analyte after the excitation light has been irradiated on the analyte, and a second detector configured to detect the fluorescence generated from the analyte after the excitation light has been irradiated on the analyte. The second detector detects the fluorescence with a measurement range that is different from a measurement range of the first detector. The first detector and the second detector detect the fluorescence simultaneously.

### Advantageous Effect of the Invention

According to an aspect of the present invention, a fluorescence detection apparatus may be provided that can simultaneously measure a plurality of analyte components having different concentrations within a wide measurement range.

### BRIEF DESCRIPTION OF THE DRAWINGS

FIG. 1 is a schematic diagram of a two-dimensional HPLC system according to a first embodiment of the present invention;

FIG. 2 is a flowchart illustrating a second dimension fluorescence detection process according to an embodiment of the present invention;

FIG. 3 is a flowchart illustrating a conventional fluorescence detection process;

FIG. 4 is a schematic flow chart of Comparative Example 1;

FIG. 5 is a schematic flowchart of Comparative Example 2;

FIG. 6A is an upper perspective view of a second dimension fluorescence detection apparatus according to an embodiment of the present invention;

FIG. 6B is a schematic top view of the second dimension fluorescence detection apparatus according to an embodiment of the present invention;

FIG. 7A shows a top view, a left side perspective view, a right side perspective view, and a bottom view of a cell included in the second dimension fluorescence detection apparatus of FIG. 6A;

FIG. 7B is a perspective view of the cell shown in FIG. 7A;

FIG. 8 is a view of the fluorescence detection apparatus with a first detector and a second detector removed;

FIG. 9 is a block diagram of a fluorescence detector according to Comparative Example 3;

FIG. 10 is a block diagram of a fluorescence detector according to an embodiment of the present invention;

FIG. 11A is an upper perspective view of a first dimension fluorescence detection apparatus included in the system of FIG. 1;

FIG. 11B is a top schematic view of the first dimension fluorescence detection apparatus included in the system of FIG. 1;

FIG. 12A shows a top view, a left side perspective view, a right side perspective view, and a bottom view of a cell provided in the first dimension fluorescence detection apparatus of FIG. 11A;

FIG. 12B is a perspective view of the cell shown in FIG. 12A;

FIG. 13 shows a configuration example of an integrated detection apparatus in which a first dimension fluorescence detection apparatus and a second dimension fluorescence detection apparatus are integrated;

FIG. 14 is a schematic diagram of a system in which an MS apparatus and a PC are connected to a two-dimensional HPLC system according to a second embodiment of the present invention;

FIG. 15 is a schematic diagram of an HPLC system including the fluorescence detection apparatus shown in FIG. 6B according to a third embodiment of the present invention;

FIG. 16 is a schematic diagram of an HPLC system including two of the fluorescence detection apparatuses shown in FIG. 6B according to a fourth embodiment of the present invention;

FIG. 17A shows detection results of detecting D-serine and L-serine using a range for high concentration detection;

FIG. 17B shows detection results of detecting D-serine and L-serine using a range for low concentration detection;

FIG. 18A shows results of a linearity test performed with respect to the serine detection results obtained by the fluorescence detection apparatus using an LED according to an embodiment of the present invention;

FIG. 18B shows results of a linearity test performed with respect to serine detection results obtained by a fluorescence detection apparatus using a xenon lamp according to a comparative example;

FIG. 19A shows a sensitivity comparison of low concentration range detections performed by the fluorescence detection apparatus using the LED according to an embodiment of the present invention and the fluorescence detection apparatus using the xenon lamp according to the comparative example in detecting the concentration of L-serine in black vinegar;

FIG. 19B shows a sensitivity comparison of high concentration range detections performed by the fluorescence detection apparatus using the LED according to an embodiment of the present invention and the fluorescence detection apparatus using the xenon lamp according to the comparative example in detecting the concentration of D-serine in black vinegar;

FIG. 20 shows mass spectrometry analysis results (QDa data) and fluorescence detection results of alanine (Ala);

FIG. 21 shows mass spectrometry analysis results and fluorescence detection results of leucine (Leu);

FIG. 22 shows mass spectrometry analysis results and fluorescence detection results of aspartic acid (Asp);

FIG. 23 shows mass spectrometry analysis results and fluorescence detection results of glutamic acid (Glu);

FIG. 24A shows a result of a linearity test performed with respect to high concentration range detection results of fluorescein obtained using the system of FIG. 16;

FIG. 24B shows a result of a linearity test performed with respect low concentration range detection results of fluorescein obtained using the system of FIG. 16;

FIG. 25A shows a result of a linearity test performed with respect to high concentration range detection results of fluorescein obtained by the fluorescence detection apparatus using a xenon lamp according to a comparative example;

FIG. 25B shows a result of a high sensitivity linearity test performed with respect to the detection range of the fluorescein detection performed by the fluorescence detection apparatus using a xenon lamp according to the comparative example;

FIG. 26A shows detection results of detecting 1,000 ng/mL of fluorescein under conditions of a sampling rate of 5 Hz and a time constant of 2 sec using the system of FIG. 16 according to an embodiment of the present invention;

FIG. 26B shows detection results of detecting 1,000 ng/mL of fluorescein under conditions of a sampling rate of 2 Hz and a time constant of 5 sec using the system of FIG. 16 according to an embodiment of the present invention;

FIG. 27A shows detection results of detecting 1,0000 ng/mL of fluorescein under conditions of a sampling rate of 5 Hz and a time constant of 2 sec using the system of FIG. 16 according to an embodiment of the present invention; and

FIG. 27B shows detection results of detecting 1,0000 ng/mL of fluorescein under conditions of a sampling rate of 2 Hz and a time constant of 5 sec using the system of FIG. 16 according to an embodiment of the present invention.

### EMBODIMENTS FOR IMPLEMENTING THE INVENTION

In the following, embodiments of the present invention are described with reference to the accompanying drawings.

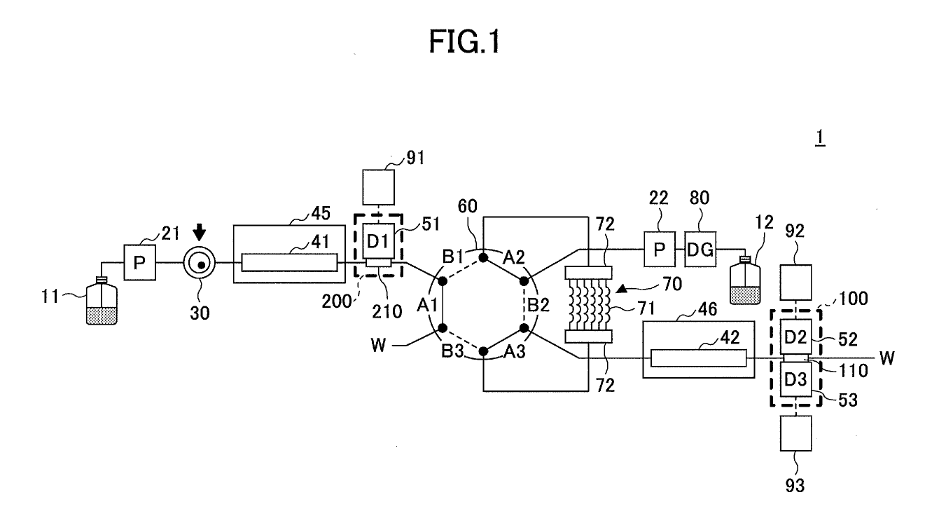

First, a system configuration of a liquid chromatography apparatus 1 as an embodiment of a two-dimensional HPLC system according to the present invention will be described with reference to FIG. 1.

In the following, a liquid chromatography apparatus implementing two-dimensional (two-step) separation (separation by type of amino acid, separation of optical isomers), first dimension detection, and second dimension detection will be described as an example. However, the present invention can be applied to any form of chromatography apparatus, including one-dimensional and multi-dimensional chromatography apparatuses, as long as it includes a component implementing functions of a fluorescence detection apparatus 100 according to an embodiment of the present invention. Note that examples of a one-dimensional chromatography apparatus will be described with respect to a third embodiment and a fourth embodiment of the present invention.

Also, in the following, the fluorescence detection apparatus 100 is described in connection with the detection of optical isomers of an amino acid. However, the fluorescence detection apparatus 100 according to the present invention is not limited to the detection of optical isomers of an amino acid, but can be applied to the detection of a plurality of analyte components contained at a wide range within a sample, particularly, those having an abundance ratio ranging over 5 orders of magnitude.

[Two-Dimensional HPLC System (Liquid Chromatography Apparatus)]

The liquid chromatography apparatus 1 according to one embodiment of the present invention shown in FIG. 1 includes a first mobile phase supply unit 11, a first pump 21, a sample injection unit 30, a reversed phase column 41, a reversed phase column thermostatic chamber 45, a first dimension detector 51, a first dimension data processing unit 91, a flow path switching valve 60, a multi-loop unit 70, a second mobile phase supply unit 12, a degassing apparatus 80, a second pump 22, a chiral column 42, a chiral column thermostatic chamber 46, a first detector 52 and a second detector 53 as a second dimension detector, and a first data processing unit 92 and a second data processing unit 93 as a second dimension data processing unit.

The first mobile phase supply unit (tank or container) 11 stores a first liquid as a mobile phase. The first liquid is transferred by the first pump 21, and the sample injection unit (autosampler) 30 that is provided on the flow path injects a sample containing components having optical isomers, such as plural types of amino acids or amino acid derivatives, for example. The sample, together with the first liquid, is transferred to the reversed phase column 41 that is maintained at a constant temperature in the reversed phase column thermostatic chamber 45.

The plurality of components contained in the sample, such as amino acids or amino acid derivatives are separated from each other by the reversed phase column 41, eluted at different times, and detected by the first dimension detector (D1) 51.

A signal of a component of the sample detected by the first dimension detector 51 is subjected to data processing by the first dimension data processing unit 91, and a chromatogram can be drawn based on information on the signal intensity corresponding to the elution time and amount of the separated component.

Also, the second mobile phase supply unit 12 stores a second liquid as a mobile phase. The degassing apparatus (DG) 80 for degassing may be provided downstream of the second mobile phase supply unit 12.

Also, the flow path switching valve 60 switches the flow paths of the first liquid and the second liquid between a combination of A1, A2 and A3 (represented by solid lines) and a combination of B1, B2 and B3 (represented by the dotted lines) as shown in FIG. 1.

When a component of a sample, such as an amino acid or an amino acid derivative, is detected by the first dimension detector 51, only a fraction containing the detected component of the sample, such as the amino acid or the amino acid derivative detected by the first dimension detector 51, can be transferred to the multi-loop unit 70 together with the first liquid by switching the flow path from the combination of A1, A2 and A3 to the combination of B1, B2 and B3 only when the fraction containing the component passes through the flow path switching valve 60.

The multi-loop unit 70 includes a plurality of loops 71 and switching means 72 for connecting a selected loop selected from among the plurality of loops 71 to the flow paths B1 and B3. By using such a multi-loop unit 70, each of the fractions containing the components of the sample that have been separated by the reversed phase column 41 may be separately held in a loop 71. That is, by switching the loop 71 connected to the flow paths B1 and B3 in accordance with the detection of a component of the sample by the first dimension detector 51, each loop 71 may be arranged to hold a different component of the sample.

By switching the switching means 72, a desired component of the sample that is held in one of the plurality of loops 71 can be transferred together with the second liquid to the chiral column 42, which is maintained at a constant temperature by the column thermostatic chamber 46. In the chiral column 42, optical isomers (e.g., D-form and L-form enantiomers of amino acids or the amino acid derivatives) in the sample transferred thereto are separated from each other.

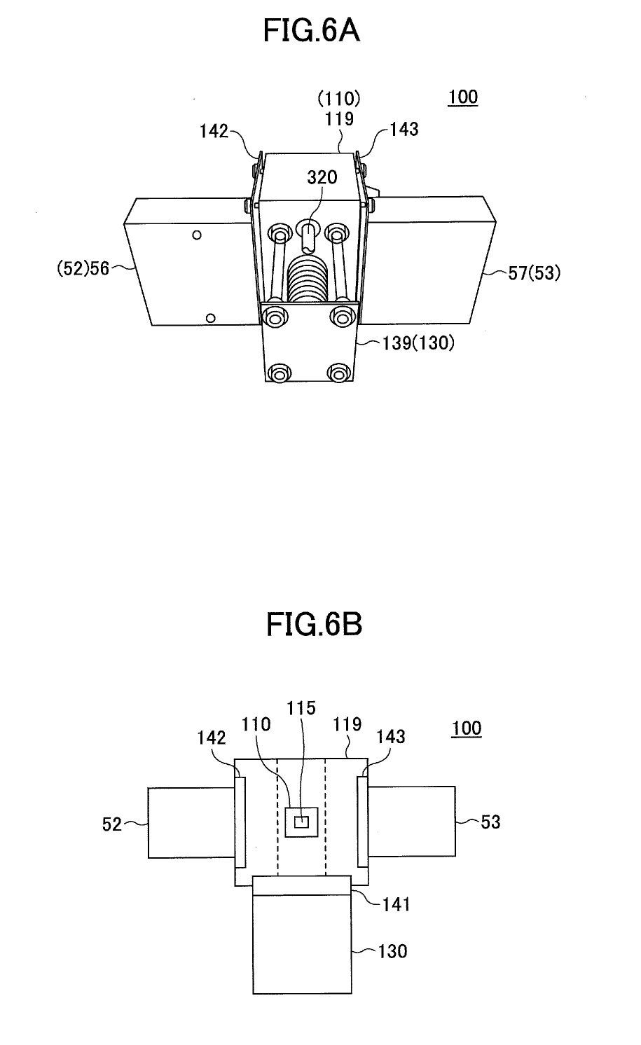

The optical isomers of the component of the sample that have been separated from each other elute at different times and are detected by the first detector 52 and the second detector 53. Note that the configurations and detection methods of the first detector (D2) 52 and the second detector (D3) 53 included in the second dimension detector implementing the fluorescence detection apparatus 100 according to an embodiment of the present invention are described below with reference to FIGS. 6A to 8 and FIG. 10. The first detector 52 and the second detector 53 are provided for the same cell and are configured to perform detection simultaneously with different sensitivities.

By switching between the loops 71 using the switching means 72, separation of optical isomers and detection by the second dimension detectors 52 and 53 can be sequentially performed in the chiral column 42 with respect to each component held in the loop 71. In this way, separation of optical isomers and detection can be performed with respect to all the components held in the multi-loop unit 70.

When the components contained in a sample are amino acids, all the amino acids can by analyzed at once in one process that involves separating the sample as a mixture of D-form and L-form amino acids in the reversed phase column 41, passing the plural types of amino acids through the plurality of loops 71, and sequentially introducing the plurality types of amino acids to the chiral column 42 to separate and quantitate the optical isomers of the plural types of amino acids.

Note that a degassing apparatus may be provided upstream of the pump 21 for the purpose of reducing the influence of the gas contained in the mobile phase. Also, a plurality of mobile phases/columns and means for switching between such mobile phases/columns may be added as appropriate according to analysis conditions of the component to be analyzed.

In the present system, the second dimension detection system used for the separation of optical isomers as described above is referred to as a fluorescence detection system according to an embodiment of the present invention. The fluorescence detection system includes a column and a column thermostatic chamber as a separation unit, and a fluorescence detector, a data processing unit, and the like as a fluorescence detection apparatus.

Further, the fluorescence detection system includes a sample injection unit, a multi-loop unit, and flow path switching valves provided in these units that function as a sample loading unit of the fluorescence detection system.

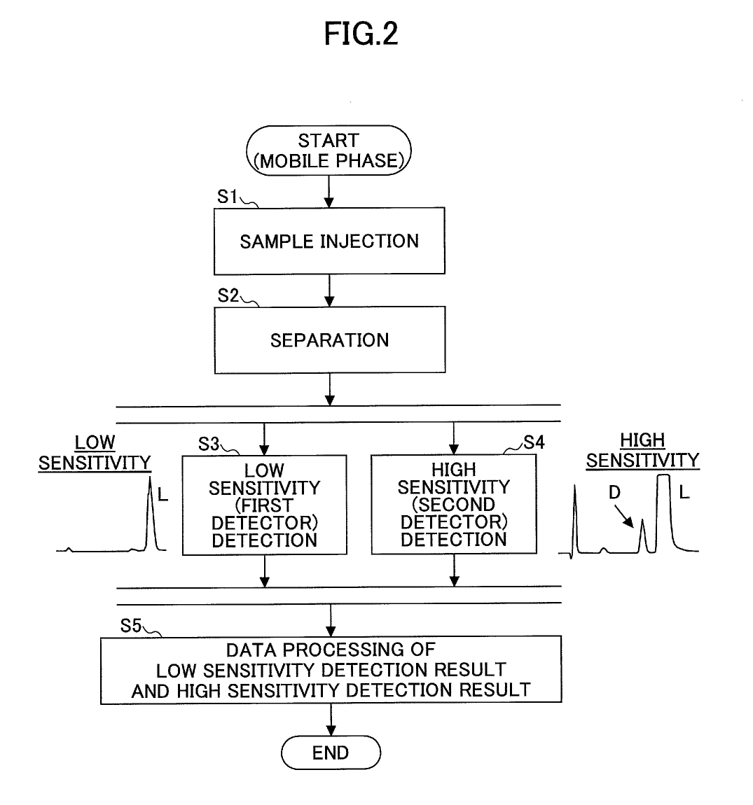

FIG. 2 is a flowchart showing a detection process for detecting optical isomers as an example process relating to the fluorescence detection system according to the present embodiment.

In step S1 of FIG. 2, the sample loading unit injects a sample to be analyzed (e.g., a sample containing specific amino acids) into the mobile phase.

In step S2, the components in the sample are separated from each other in a column.

Then, in step S3, an analyte component passing through the flow cell of the fluorescence detector (see FIG. 6B) is detected by the first detector 52. At the same time, in step S4, the analyte component that passes through the same flow cell is detected by the second detector 53 at the same time as step S3 in which the first detector 52 detects the analyte component.

Then, in S5, the data processing units 92 and 93 concurrently perform data processing (quantitation, etc.) on detection information obtained by the first detector 52 and detection information obtained by the second detector 53 with different measurement ranges.

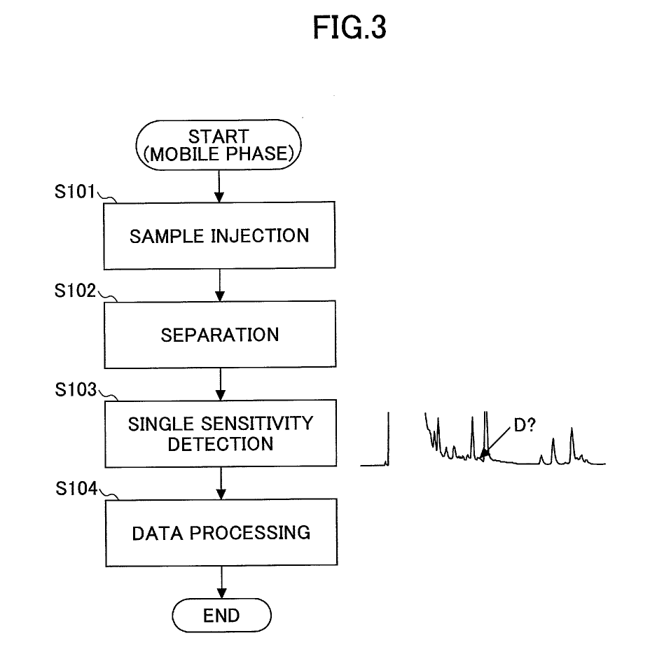

FIG. 3 is a flowchart of a detection process performed by a conventional fluorescence detection system.

In the conventional fluorescence detection system, in step S101 of FIG. 3, a sample is injected, and thereafter, in step S102, the components of the sample are separated by a column. Then, in step S103, plural analyte components in a cell with different concentrations are measured using a conventional xenon lamp fluorescence detector, for example. As such, when the abundance ratio of the plural analyte components in the sample (difference in concentration of the analyte components) exceeds the measurement range, the analysis accuracy will inevitably be substantially degraded.

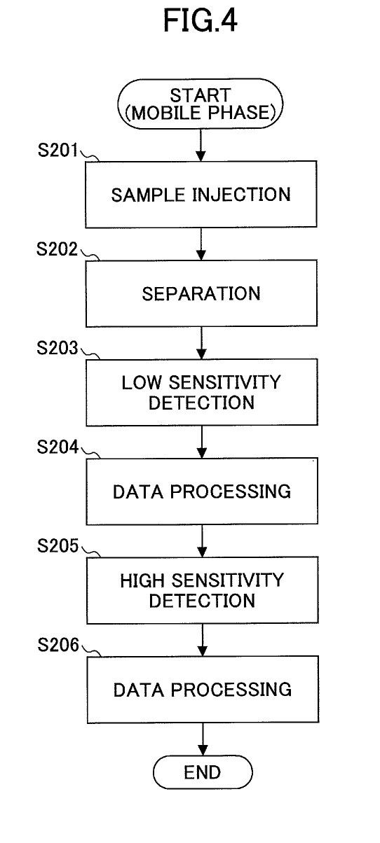

In this respect, in the process flow according to Comparative Example 1 as shown in FIG. 4, detection is performed by two types of detectors that are arranged in series (S203, S205). Although the measurement range can be widened by implementing such measure, arranging cells in two stages causes diffusion of the analyte components and degradation of analytical accuracy due to decomposition by light irradiation.

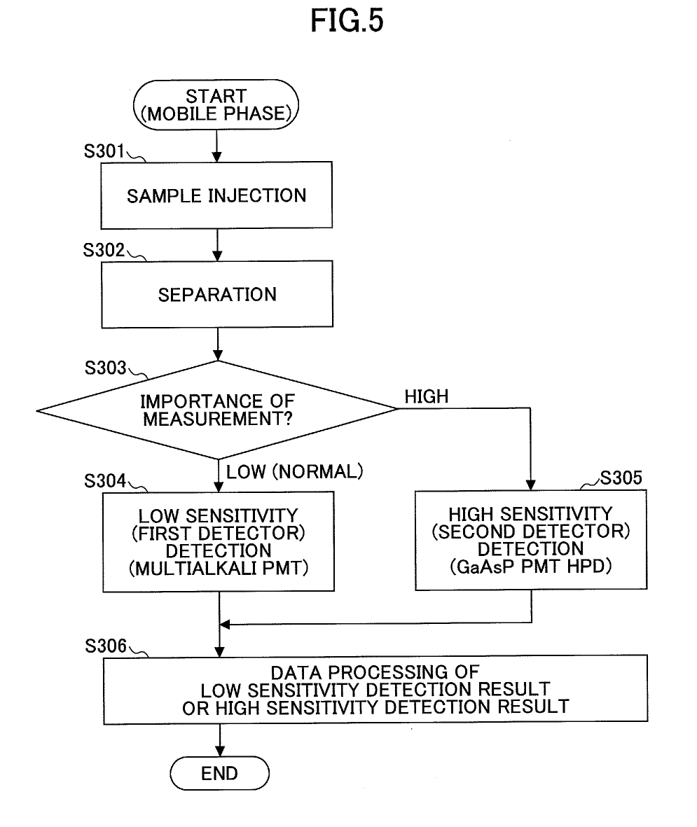

Also, in the process flow according to Comparative Example 2 as shown in FIG. 5, a sample is detected in a detection process by selecting one of two types of detectors having different sensitivities (measurement ranges) for one cell (S303, S304, S305). However, although detection can be performed at the desired sensitivity in this process flow, only one of the detectors can be used at one time, and a detection result can only be output by one detector (S306). In addition, since two types of detectors are alternatively switched and used, a control mechanism for switching between the detectors is required.

In comparison to the above process flows of FIGS. 3 to 5, in the process flow according to an embodiment of the present invention as shown in FIG. 2, in steps S3 and S4, two components with different concentrations can be simultaneously measured using two detectors with a wide measurement range. Also, because detection can be performed simultaneously with two detectors using one cell, cells do not have to be arranged in two stages in order to widen the measurement range as in the conventional example shown in FIG. 5, and as such, degradation of analysis accuracy due to diffusion/decomposition of the analyte components may be avoided.

[Fluorescence Detection Apparatus]

FIGS. 6A and 6B show the configuration of the fluorescence detection apparatus 100. FIG. 6A is an external view of the fluorescence detection apparatus 100, and FIG. 6B is a top view of the fluorescence detection apparatus 100 when a cell 110 and the detectors 52 and 53 are removed from the fluorescence detection apparatus 100 shown in FIG. 6A.

As shown in FIGS. 6A and 6B, the fluorescence detection apparatus 100 includes the cell 110, a light source 130, the first detector 52, and the second detector 53.

As shown in FIG. 6A, in the fluorescence detecting apparatus 100, the cell 110 is accommodated in a housing 119, the light source 130 is accommodated in a housing 139, the first detector 52 is covered by a case 56, and the second detector 53 is covered by a case 57.

Analyte components of a sample (e.g., amino acids) are introduced into the cell 110. In the present example, the cell 110 is a flow cell, and a sample flowing through a pipe 320 is introduced into the cell 110. The cell 110 is preferably made of quartz, which has high light permeability and high solution resistance. As an example, the capacity of a solution passing portion (sample passing portion) 115 of the cell 110 is 7.2 μL.

The light source 130 irradiates excitation light on the analyte components in the cell 110. In the present invention, the light source 130 is preferably configured by an LED (Light Emitting Diode). A blue LED that emits light at a wavelength of 470 nm may be used with respect to amino acids fluorescently derivatized with NBD-F.

The first detector 52 detects fluorescence generated after the excitation light is irradiated on the analyte components. At the same time, the second detector 53 detects the fluorescence with a different sensitivity (second measurement range) from that of the first detector 52. The first detector 52 and the second detector 53 are optical sensors made of photomultiplier tubes (PMTs).

As shown in FIG. 6B, the first detector 52 and the second detector 53 are arranged on (in close proximity to) the surface of the cell 110.

Further, as shown in FIG. 6B, in the fluorescence detection apparatus 100, an excitation light filter 141 in contact with the light source 130, a first fluorescence filter 142 in contact with the first detector 52, and a second fluorescence filter 143 in contact with second detector 53 are arranged in contact with the cell 110. Specifically, in the example of FIG. 6B, the cell 110 is arranged inside the cell housing 119, and the first fluorescence filter 142 and the second fluorescence filter 143 are in contact with the cell housing 119, in close proximity to the cell 110. However, in other examples, the filters 141, 142, and 143, the detectors 52 and 53, the light source 130 and the like may be arranged to be in direct contact with the cell 110 (surface).

Note that the expressions “arranged in contact with the cell” or “arranged on the surface of the cell” in the above description is to be construed to encompass both a state where the filters 141, 142, and 143, the detectors 52 and 53, the light source 130, and the like are arranged directly in contact with the cell 110 (surface) and a state where the elements are arranged in close proximity to the cell 110 (surface) as shown in FIG. 6B.

The light source 130 emits light of a predetermined wavelength (e.g., 470 nm), for example. The excitation light filter 141 is arranged on an incident surface of the cell 110 (bottom side of FIG. 6B) and is configured to pass light with a desired wavelength range centered around 470 nm in consideration of manufacturing tolerances and the like so that the sample is irradiated with light having the predetermined wavelength.

When the sample in the cell 110 is irradiated with light having the predetermined excitation wavelength and excited, the sample generates fluorescence upon returning to the ground state. The first fluorescence filter 142 passes fluorescence having a predetermined fluorescence wavelength (e.g., 512 nm corresponding to NBD-amino acid) that is generated from the sample.

The second fluorescence filter 143 passes the same wavelength as the wavelength passed by the first fluorescence filter 142.

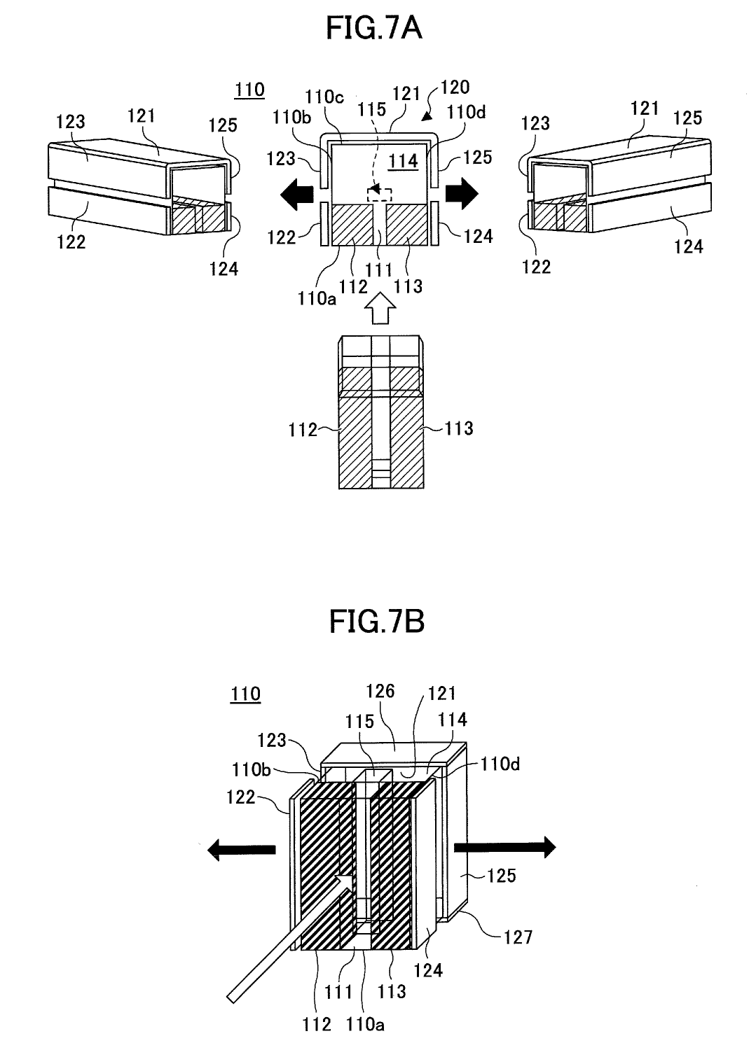

Note that the first detector 52 and the second detector 53 are arranged on surfaces of the cell 110 that are different (face different directions) from the incident surface of the cell 110. In the example of FIG. 6B, the first detector 52 is arranged on a first surface 110b of the cell 110 that is lateral to the incident surface 110a of the cell 110 on which excitation light is incident (see FIG. 7A). The second detector 53 is arranged on a second surface 110d of the cell 110 that is at the other lateral side of the incident surface 110a and is symmetrical to the first surface 110b.

As shown in FIGS. 6A and 6B, the light source (LED) 130, the excitation light filter 141, the cell 110, the first fluorescence filter 142, the first detector 52, the second fluorescence filter 143, and the second detector 53 are integrally connected to each other.

With such a configuration, when a sample in the cell 110 is irradiated with light, fluorescence generated from the sample can be simultaneously introduced into the first detector 52 and the second detector 53.

With such a configuration, simultaneous detection can be performed under the same conditions with two measurement ranges by installing two detectors (PMT sensors) 52 and 53 with different sensitivities in one cell 110. In this way, the measurement range can be widened. For example, in the case of detecting NBD-amino acids, quantitation over a wide range from a detection lower limit of 500 [amol] to a detection upper limit of 500 [pmol] may be possible.

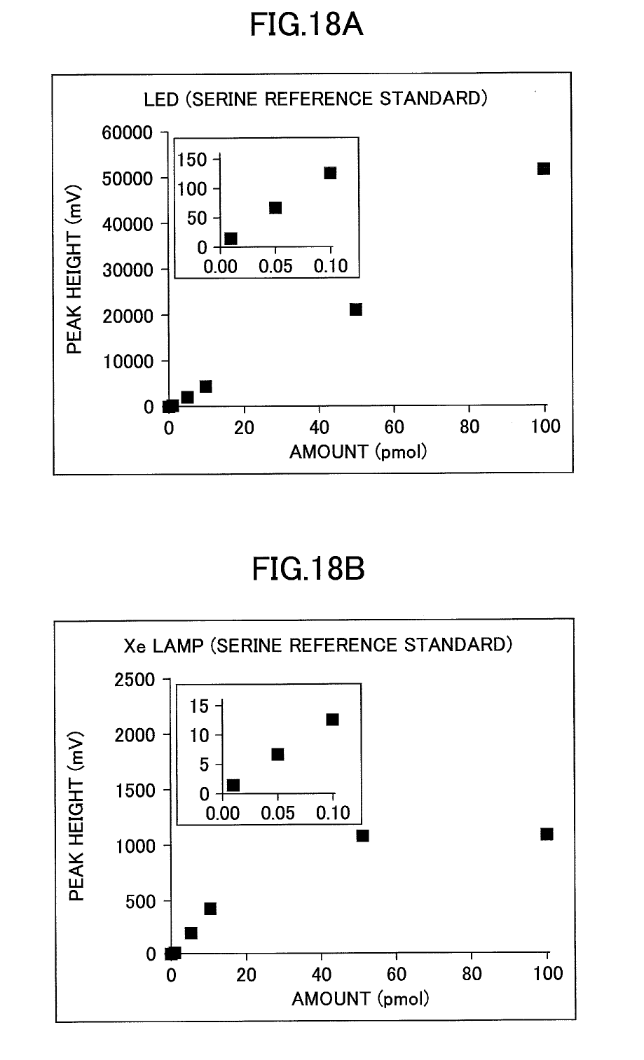

Specifically, for example, when a NBD-serine reference standard was analyzed using the fluorescence detection apparatus 100 as described below with respect to Example 2, the analysis results showed good linearity over a range extending at least 6 orders of magnitude (FIGS. 18A and 18B). In this way, one single fluorescence detection apparatus 100 can be used to cover the measurement range (quantitative range) of protein-constituting D-form and L-form amino acids (43 types) contained in a biological/food sample at various concentrations within a wide concentration range.

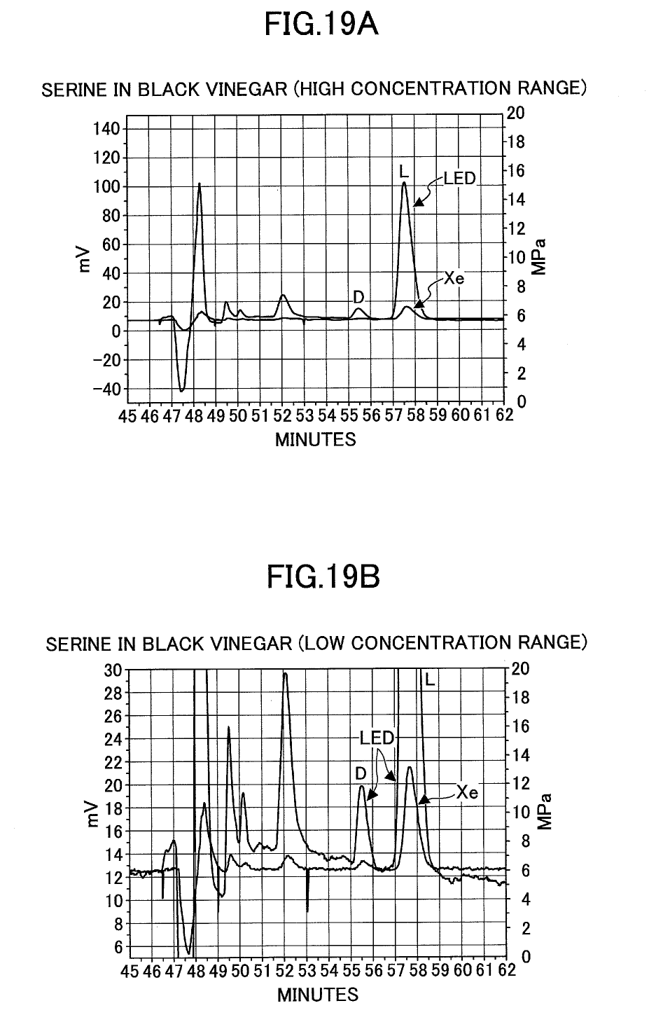

For example, the concentration of D-serine among the types of serine (Ser) contained in black vinegar is several percent. As described below with respect to Example 3, even when the abundance ratios of L-serine and D-serine contained in black vinegar differ by at least 3 orders of magnitude, both the L-serine and the D-serine can be simultaneously detected by installing two detectors having different measurement ranges (FIGS. 19A and 19B). Note that application of such technique for widening the measurement range is not limited to the detection of amino acids.

In the following, the configuration of the cell 110 used in the fluorescence detection apparatus 100 will be described with reference to FIGS. 7A and 7B. FIG. 7A shows a top view (center), a left side perspective view, a right side perspective view, and a bottom view of the cell 110 provided in the fluorescence detection apparatus 100, and FIG. 7B shows a front perspective view of the cell 110.

The solution passing portion 115 for holding the sample is formed at a center portion of the cell 110. The cell 110 is composed of transparent quartz blocks 111 and 114, and black quartz blocks 112 and 113.

Specifically, as can be appreciated from FIGS. 7A and 7B, the transparent quartz block 111 is arranged at a light incidence location toward the light source 130 where the excitation light is incident. The black quartz blocks 112 and 113 are bonded to the side ends of the transparent quartz block 111 on the incident surface 110a.

The transparent quartz block 114 is arranged at fluorescence emitting locations toward the first detector 52 and the second detector 53 at which fluorescence generated from a sample is emitted to the first detector 52 and the second detector 53.

A reflection mirror is deposited on the surfaces lateral to and opposing the incident surface 110a. More specifically, in FIGS. 7A and 7B, the reflection mirror includes a mirror portion 121 arranged on an opposing surface 110c opposing the incident surface 110a, mirror portions 122 and 123 arranged on portions of the first surface 110b other than a portion where fluorescence is passed, and mirror portions 124 and 125 arranged on portions of the second surface 110d other than a portion where fluorescence passes.

Further, in order to collect more light from the sample, mirror portions 126 and 127 may be arranged at least on portions of the upper surface and the lower surface of the cell 110 that reflect light.

By configuring the cell 110 in this manner, a lens may not have to be installed for the purpose of collecting light such that the light source 130 and the cell 110 may be arranged in direct contact (or in close proximity) with each other, and the cell 110 and the second dimension detectors 52 and 53 may be arranged in direct contact (or in close proximity) with each other.

Note that in this configuration example, two second dimension detectors 52 and 53 are provided in the cell 110 that has a quadrilateral shape. However, the shape of the cell 110 is not limited to a quadrilateral shape, and may alternatively be a triangle, a pentagon, a hexagon, or some other polygon as long as fluorescence generated from the sample in the cell 110 can enter the two detectors 52 and 53 under the same conditions.

Further, the number of detectors provided for one cell may be more than two if the fluorescence generated from the sample in the cell 110 can enter the detectors under the same conditions through reflection or the like. For example, when the cell 10 has a hexagonal shape, the cell 110 may be provided with four detectors, and appropriate adjustments may be made according to the required measurement range and sensitivity.

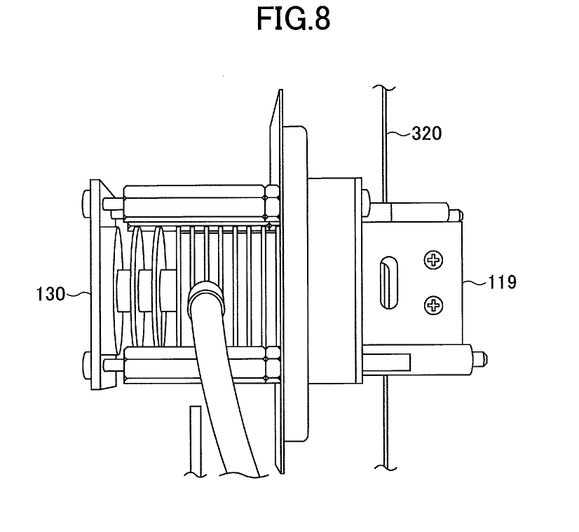

FIG. 8 shows the housing 119 and the light source 130 of the cell 110 in a state where the second dimension detectors 52 and 53 are removed from the fluorescence detection apparatus 100 shown in FIG. 7A.

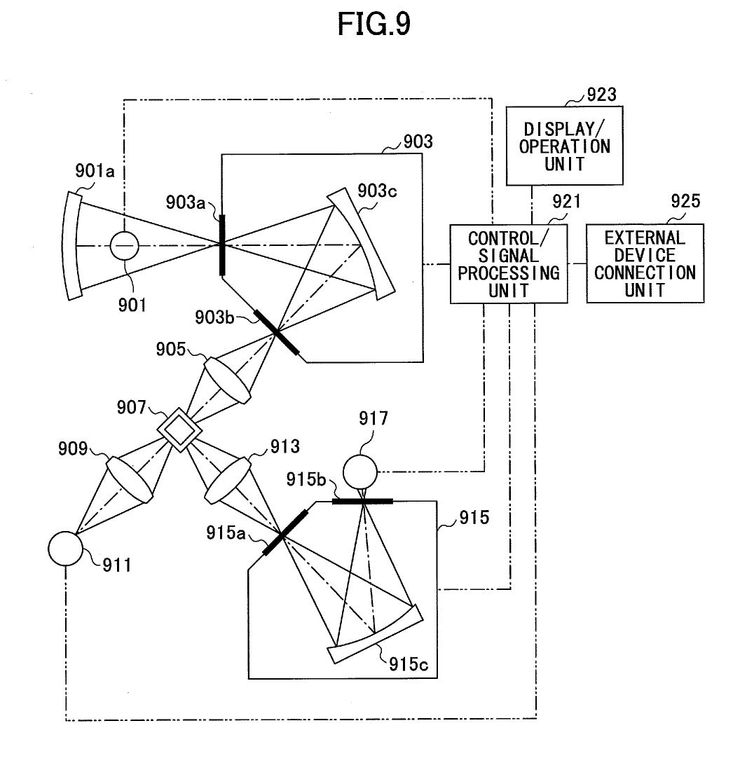

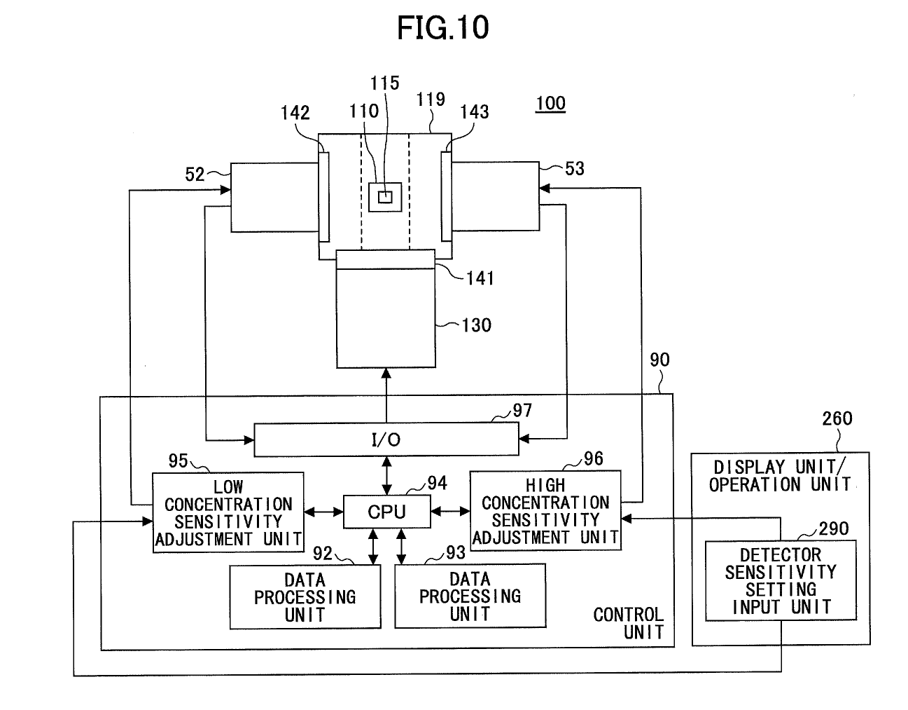

FIG. 9 is a control block diagram showing a configuration of a fluorescence detection apparatus according to Comparative Example 3. FIG. 10 is a block diagram showing a control system configuration of the fluorescence detection apparatus 100 according to an embodiment of the present invention.

Conventionally, a fluorescence detection apparatus uses a xenon lamp, which has a wide wavelength selection range and can be utilized as a light source in various fields including but not limited to fluorescence detection.

However, because the lifetime of a xenon lamp may be as short as 500 hours (20 days) and the xenon lamp may be unstable after replacement, significant loss time has been an obstacle to continuous multi-analyte analysis. Also, because the light quantity of the xenon lamp becomes unstable due to environmental factors (e.g., ambient temperature), using the xenon lamp may require high maintenance costs for environmental maintenance, such as air conditioning and the like. Further, the xenon lamp poses other problems, such as high power consumption and ozone generation, for example.

As can be appreciated from FIG. 9 showing the configuration of the fluorescence detection apparatus according to Comparative Example 3, in the case of performing detection using a xenon lamp, slits 903a and 903b, a grating 903c, a lens 905, and the like are arranged between a light source 901 and a flow cell 907 for enabling spectroscopic analysis, and a predetermined distance is provided between the light source 901 and the flow cell 907. Also, slits 915a and 915b, a grating 915c, a lens 913, and the like are arranged between a detector 917 and the flow cell 907, and as such, a predetermined distance has to be provided between the detector 917 and the flow cell 907. Thus, it is difficult to provide a plurality of detectors for one flow cell.

Further, to address the problem of light quantity instability of the xenon lamp due to environmental factors, a structure for detecting the light quantity and correcting the detection has been conceived. Specifically, in FIG. 9 showing the configuration according to Comparative Example 3, in addition to the photodetector for detecting fluorescence emitted from a sample in a sample cell, a photodetector 911 for measuring scattered light from the sample at the same site as the fluorescence measurement site of the sample is provided on the opposite side of the sample cell.

However, attempting to address the problem of light quantity instability of the xenon lamp as described above results in an increase in the number of required components and enlargement of the apparatus.

In this respect, as shown in FIGS. 6A to 8 and FIG. 10, the fluorescence detection apparatus 100 according to an embodiment of the present invention includes the light source 130 that uses an LED, which has a substantially longer lifetime (e.g., 25000 hours) than a xenon lamp. As such, the fluorescence detection apparatus 100 according to the present embodiment may be capable of continuous operation for approximately 50 times longer than the fluorescence detection apparatus using a xenon lamp. Further, by using an LED as the light source, power consumption may be lowered (to about several watts) to achieve approximately a 96%-reduction in power consumption as compared with the apparatus using the xenon lamp, and its impact on the natural environment can be improved. Also, because LEDs do not generate ozone, a clean environment can be maintained.

Further, according to an aspect of the present invention, to address the above problem of apparatus enlargement, an LED is used as a light source with an excitation wavelength fixed to 470 nm for NBD. Thus, an LED that can narrow down light emission to a specific wavelength band can be used. By using an LED, diffraction gratings and lenses for spectroscopy may be omitted because, owing to characteristics of the LED, a wavelength may show a sharper peak at a specific value as compared with the case of using a xenon lamp. In this way, the apparatus can be miniaturized.

Thus, the LED constituting the light source can be arranged close to the flow cell corresponding to the detection target without requiring spectroscopy.

Note that in consideration of manufacturing variations and the like in the LED, a filter that can be arranged in contact with the LED may be provided to improve reproducibility. The filter may be a band-pass filter or the like that specifies a wavelength band to be transmitted.

Also, because the light quantity characteristic of the LED is stable with respect to the external environment, the light quantity from the light source does not have to be referenced/corrected in the present embodiment. As such, a control mechanism in the system according to the present embodiment can be simplified as compared with the apparatus using a xenon lamp according to Comparative Example 3 as shown in FIG. 9.

For example, as shown in FIG. 10, according to an aspect of the present invention, the light quantity from the light source is not referenced/corrected at an external device connection unit (control unit) 90, and the detectors 52 and 53 are independently controlled by a CPU 94 via an interface (I/O) 97. As described below, the sensitivities of the detectors 52 and 53 can be set to different predetermined measurement ranges (sensitivities) by sensitivity adjustment units 95 and 96. Also, a display unit/operation unit 260 connected to the control unit 90 may be included in a computer 400 that is connected to the liquid chromatography apparatus 1 according to a second embodiment.

Also, according to an embodiment of the present invention, an LED that emits light with a wavelength of 470 nm is used in order to perform detection dedicated to NBD-amino acids and the like.

Thus, a sample detected by the fluorescence detection apparatus 100 according to an embodiment of the present invention that includes an LED configured to emit light at a specific wavelength is preferably a sample containing amino acids or amino acid derivatives including optical isomers.

For example, the sample may contain an L-form amino acid or amino acid derivative and a D-form amino acid or amino acid derivative having configurations as indicated below.

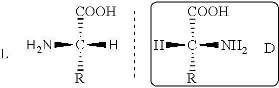

The first detector 52 detects the L form amino acid or amino acid derivative as a first component (first analyte component) with a first sensitivity (low sensitivity), i.e., first measurement range. The second detector 53 detects the D-form amino acid or amino acid derivative as the second component (second analyte component) with a second sensitivity (high sensitivity) higher than the first sensitivity, i.e., second measurement range.

Note that although the fluorescence detection apparatus using an LED according to the above-described embodiment of the present invention is applied to a second dimension fluorescence detection apparatus, the fluorescence detection apparatus using an LED can also be applied to a first dimension fluorescence detection apparatus.

[First Dimension Fluorescence Detection Apparatus]

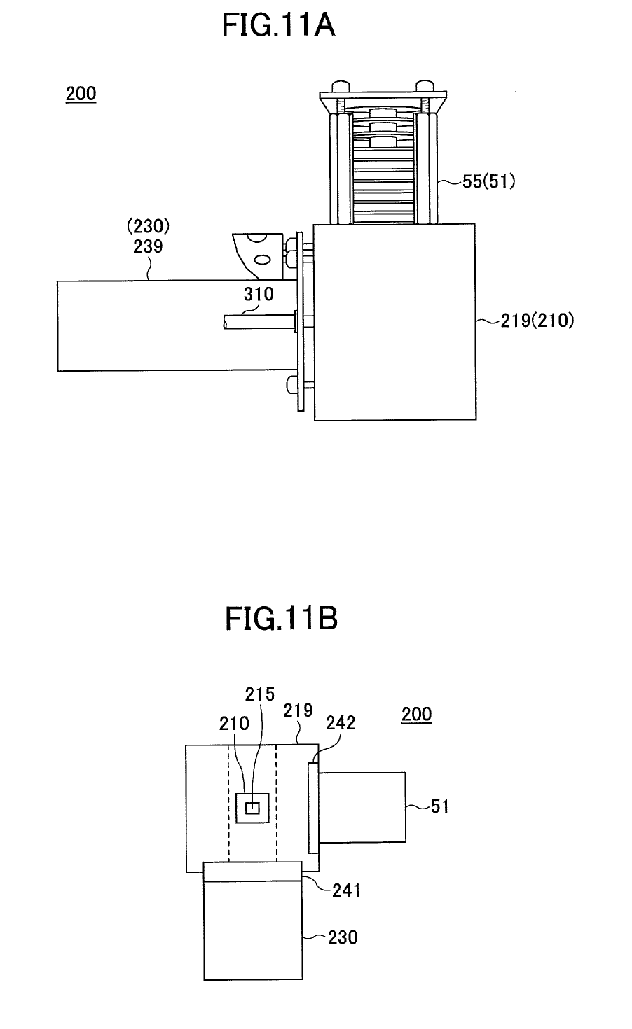

FIG. 11A is an external view of a first dimension fluorescence detection apparatus 200, and FIG. 11B is a top view of the first dimension fluorescence detection apparatus 200 when a case 55 of the fluorescence detection apparatus 200 shown in FIG. 11A is removed.

As shown in FIGS. 11A and 11B, the fluorescence detection apparatus 200 includes a cell 210, a light source 230, and a first dimension detector 51. As shown in FIG. 11A, in the fluorescence detection apparatus 200, the cell 210 is surrounded by a housing 219, the light source 230 is surrounded by a housing 239, and the first dimension detector 51 is covered by the case 55.

As with the fluorescence detection apparatus 100 described above, the light source 230 that irradiates excitation light on a sample in the cell 210 is preferably configured by an LED.

The first dimension detector 51 measures fluorescence generated from the sample when the sample is irradiated with excitation light. In the first dimension detection, the detection target corresponds to a specific substance (e.g., amino acid). The first dimension detection differs from the second dimension detection in that the cell 210 is provided with one single detector in the case where an enhanced range is unnecessary.

Also, as shown in FIG. 11B, in the fluorescence detection apparatus 200, an excitation light filter 241 that is in contact with the light source 230, and a fluorescence filter 242 that is in contact with the first dimension detector 51 are arranged in contact with (in close proximity to) the cell 210.

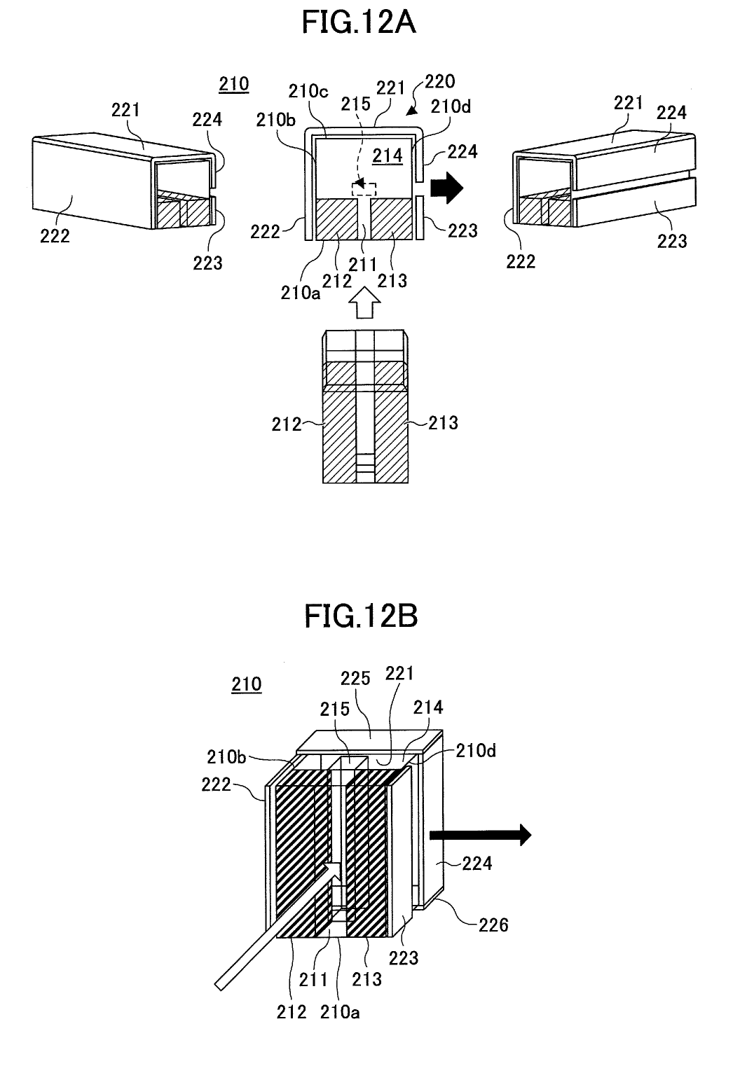

In the following, the configuration of the flow cell 210 used in the first dimension fluorescence detection apparatus 200 will be described. FIG. 12A shows a top view, a left side perspective view, a right side perspective view, and a bottom view of the cell 210, and FIG. 12B is a front perspective view of the cell 210.

Note that in FIGS. 12A and 12B, the cell 210 has a solution passing portion 215, transparent quartz blocks 211 and 214, and black quartz blocks 212 and 213 that hold the sample arranged therein in the same manner as the cell 110 shown in FIGS. 7A and 7B.

Note that because the fluorescence detection apparatus 200 is provided with one single detector, a reflection mirror can be deposited over a wider region as compared with the fluorescence detection apparatus 100. Specifically, as shown in FIGS. 12A and 12B, the reflection mirror of the fluorescence detection apparatus 200 similarly includes a mirror portion 221 provided on an opposing surface 210c opposing an incident surface 210a, mirror portions 223 and 224 provided on portions of a second surface 210d excluding a portion through which fluorescence passes. However, the reflection mirror of the fluorescence detection apparatus 200 differs from that of the fluorescence detection apparatus 100 in that it includes a mirror portion 22 provided on an entire surface of a first surface 210b of the cell 210.

Further, in order to collect more light from the sample, mirror portions 225 and 226 may be provided at least on portions of the upper and lower surfaces of the cell 210 that reflect light.

By processing the cells in the above-described manner, a lens may not have to be installed for the purpose of collecting light. In this way, the light source 230 and the cell 210 can be arranged in direct contact with each other, and the cell 210 and the first dimension detector 51 can be arranged in direct contact with each other.

By using LEDs as the light sources for enabling detection by the first dimension detector 51 and detection by the second dimension fluorescence detectors 52 and 53 in the system shown in FIG. 1, the overall size of the system can be reduced.

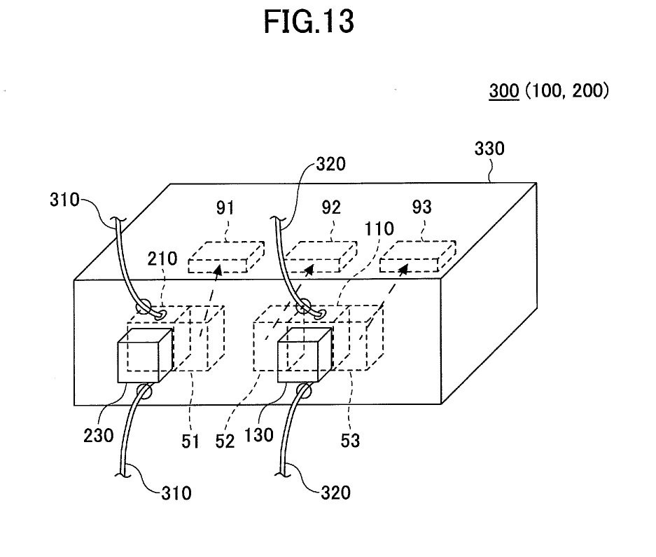

For example, as shown in FIG. 13, in some embodiments, the first dimension fluorescence detection apparatus 200, the first dimension data processing unit 91, the second dimension fluorescence detection apparatus 100 and the second dimension data processing units 92 and 93 may be integrally configured into one single apparatus. FIG. 13 shows an example configuration of an integrated detection apparatus 300 in which the above units are integrated.

[Integrated Detection Apparatus]

As shown in FIG. 13, the integrated detection apparatus 300 is covered by a case 330. By being covered by the case 330, a desired environment (temperature, humidity, luminance) can be maintained in regions where the cells 110 and 210 are disposed. Also, the processing units 91, 92, and 93 perform data processing on detection results obtained by the detectors 51, 52, and 53 such that a chromatogram can be drawn based on signal intensities according to the elution time and volume of analyte components that have been separated.

As shown in FIG. 13, the integrated detection apparatus 300 is provided with two blue LED modules as light sources 130 and 230, and three detectors 51, 52, and 53 (three channels). In one example, the control system configuration as shown in FIG. 10 may be used to switch the sensitivity (measurement range) of the detectors 51, 52, and 53.

For example, with reference to FIG. 13 and FIG. 10, the detector sensitivity setting input unit 290 of the operation unit 260 may be operated to switch settings of the detectors (PMTs) 51, 52, and 53, and the data processing units 91, 92, and 93, such as settings relating to sensitivity, the sampling rate, and the time constant between multiple stages.

In the present example, each of the three detectors is configured to independently output a 20-bit 1-V full scale output.

The sampling rate and the time constant (smoothing time) may be suitably set up according to the application by adjusting the signal/noise ratio and the resolution required for analysis, for example.

By configuring the integrated detection apparatus 300 in the above-described manner, the overall external shape of the apparatus can conform to the 19-inch rack standard. In Comparative Example 3 of FIG. 9, a lens has to be provided for one cell, and as such, one case is required for one cell. However, because a lens does not have to be provided in the fluorescence detection apparatuses 100 and 200 of the present invention, the first dimension cell 210 and the detector 51 can be integrated, and the second dimension cell 110 and the detectors 52 and 53 can be integrated within the respective apparatuses. As such, the fluorescence detection apparatuses 100 and 200 can be integrated into one case 330. In this way, the size of the case 330 may be reduced by half or more.

Further, because the integrated detection apparatus 300 has a power supply compatible with AC voltages from 100 V to 240 V and LEDs are used for the light sources 130 and 230, power consumption may be reduced down to 50 W or less. The integrated detection apparatus 300 may also enable USB serial communication and may communicate with a PC 2 and a mass spectrometer 500, which are described below.

### Second Embodiment

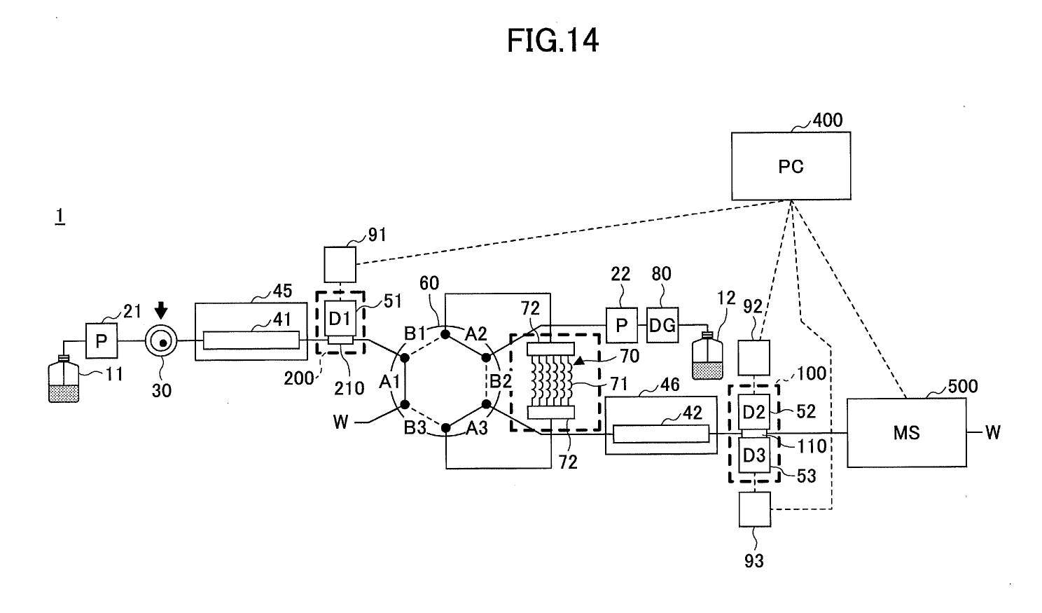

According to a second embodiment of the present invention, as shown in FIG. 14, a computer 400 and a mass spectrometer (MS apparatus) 500 may be connected to the above-described liquid chromatography apparatus 1 corresponding to the two-dimensional HPLC system according to the first embodiment of the present invention in order to enable complementary processing on detection results obtained by the two-dimensional HPLC system.

By connecting the mass spectrometer 500, detection results obtained by the fluorescence detection apparatus 100, such as detection results of L-form and D-form amino acids (fluorescence detection results), can be compared with mass spectrometry analysis results, and in this way, measurement accuracy can be improved.

According to the above-described embodiment of the present invention, a fluorescence detection apparatus can be provided that is capable of simultaneously detecting two analyte components having different concentrations using a wide measurement range (range). Further, an analysis method implemented by the above-described fluorescence detection apparatus enables simultaneous detection of two analyte components having different concentrations using a first sensitivity (first measurement range) and a second sensitivity (second measurement range), which is different from the first sensitivity.

Further, in a fluorescence detection system that includes such a fluorescence detection apparatus, detection may be enabled by a sequence of analyses that involves separating analyte components in a sample that has been transferred to a separation unit (e.g., optical isomers (D-form and L-form enantiomers of amino acids or amino acid derivatives)) from each other, and detecting the separated analyte components of the sample in the fluorescence detection apparatus using two detectors (two measurement ranges).

As described above, by using the fluorescence detection apparatus according to an embodiment of the present invention, the D-form and the L-form of an amino acid can be individually detected and their D/L compositions may be profiled so that research may be conducted, for example, on unique functions of the D-form amino acid. For example, D-serine (Ser) has functions of neuronal substance adjustment and moisture retention, D-alanine (Ala) has functions of blood glucose adjustment and skin barrier adjustment, and D-aspartic acid (Asp) has functions of hormone secretion control and anti-oxidation. As such, aspects of the present invention may be used in the medical field, for example.

Also, the fluorescence detection apparatus according to an embodiment of the present invention may be used to perform content analysis (metabolic profiling) of chiral amino acids in food as a means for taste/quality management, traceability, as well as value-added branding, for example.

### Third Embodiment

In the above-described embodiments, the fluorescence detection apparatus 100 using two detectors according to the present invention is provided as the second dimension fluorescence detection apparatus in the liquid chromatography system. However, the fluorescence detection apparatus according to an embodiment of the present invention may also be arranged in a system that does not include a configuration for fractionation.

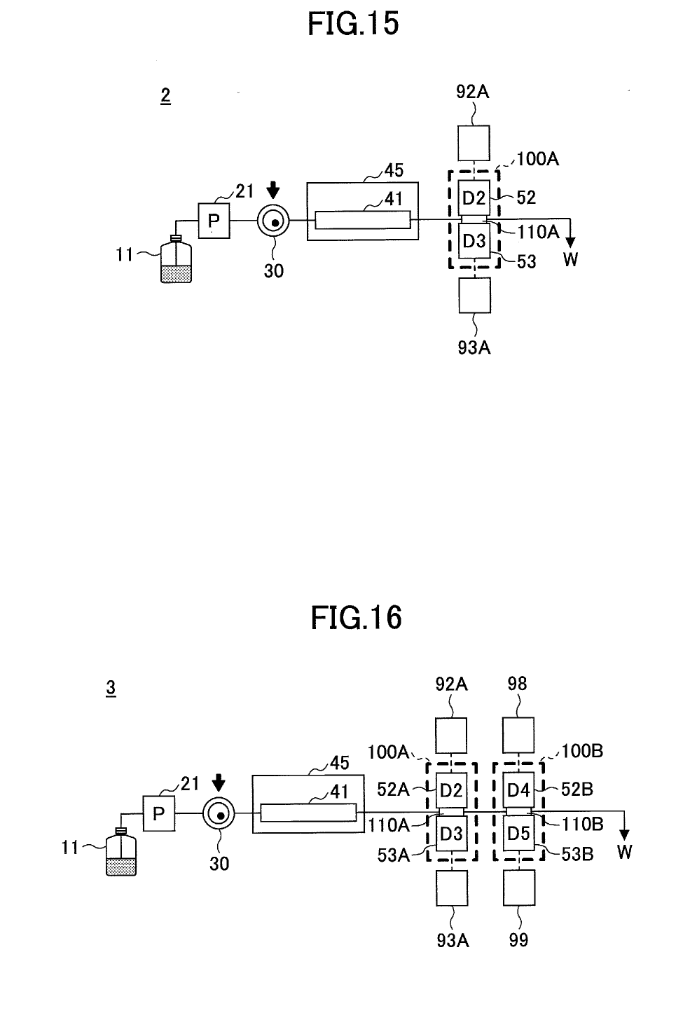

FIG. 15 is a schematic diagram of an HPLC system 2 according to a third embodiment of the present invention that includes the fluorescence detection apparatus as shown in FIG. 6B. The configuration of FIG. 15 includes a fluorescence detection apparatus 100A including detectors 52A (D2) and 53A (D3) with two different measurement ranges as a detection apparatus for performing detection after column separation.

Even in a case where the concentration of an analyte component is unknown, for example, by performing detection with a wide measurement range using two detectors, when a measurement target substance (first component) is detected, an unknown component may be simultaneously detected as a secondary detection result. For example, in detecting a complicated matrix, such as a biological sample, the measurement range is preferably widened so that a larger number of components with remarkably different concentrations can be detected.

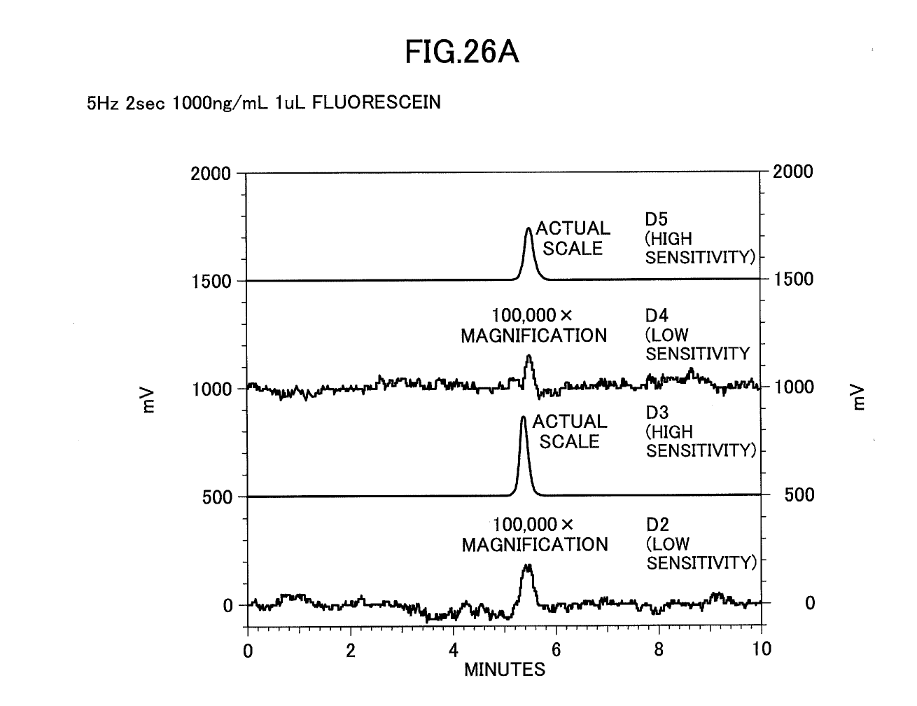

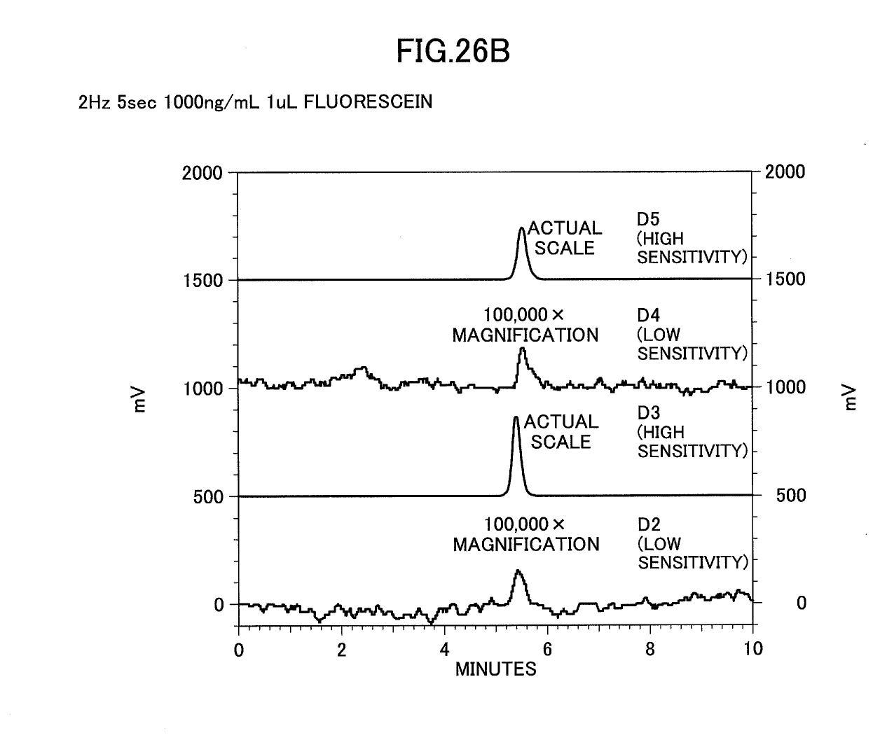

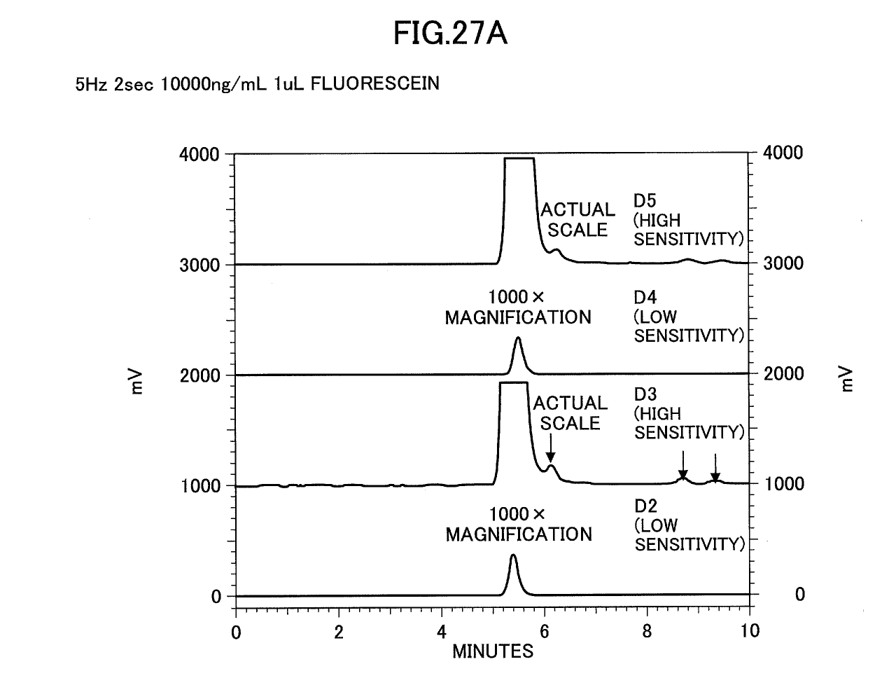

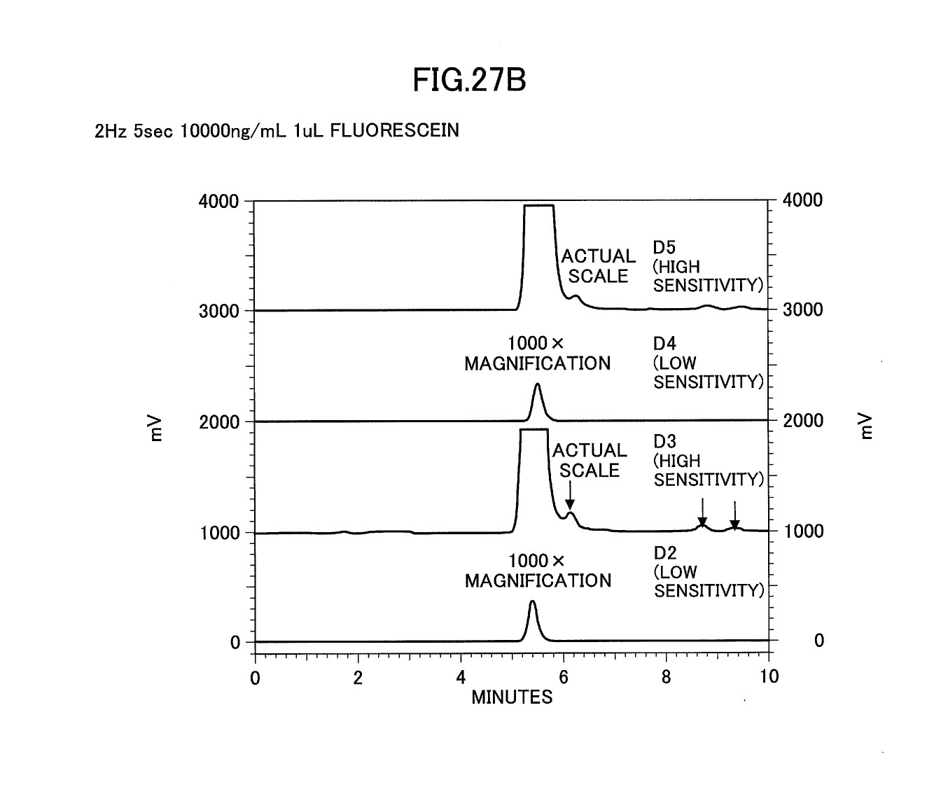

Specifically, as shown in Example 6 described below, by installing two detectors having different detection sensitivities and performing simultaneous detection, when a measurement target substance in a sample with an unknown concentration is detected (secondary mixture upon detecting fluorescein), an impurity may also be detected by low concentration range detection (see FIGS. 26A-27B).

Note that the configuration according to the present embodiment also has data processing units 92A and 93A connected to the operation unit 260 and the detector sensitivity setting input unit 290 as shown in FIG. 10 so that the sensitivities and various settings of the detectors (PMTs) 52A and 53A can be switched in stages.

For example, the sensitivity, the sampling rate, and the time constant (smoothing time) of the detectors (PMT) 52A (D2) and 53A (D3) can be suitably set up according to the application.

### Fourth Embodiment

Also, according to another embodiment, fluorescence detection apparatuses may be serially connected in an HPLC system in order to confirm reproducibility between the detection apparatuses. FIG. 16 is a schematic diagram of an HPLC system 3 according to a fourth embodiment of the present invention that includes two of the fluorescence detection apparatuses shown in FIG. 6B.

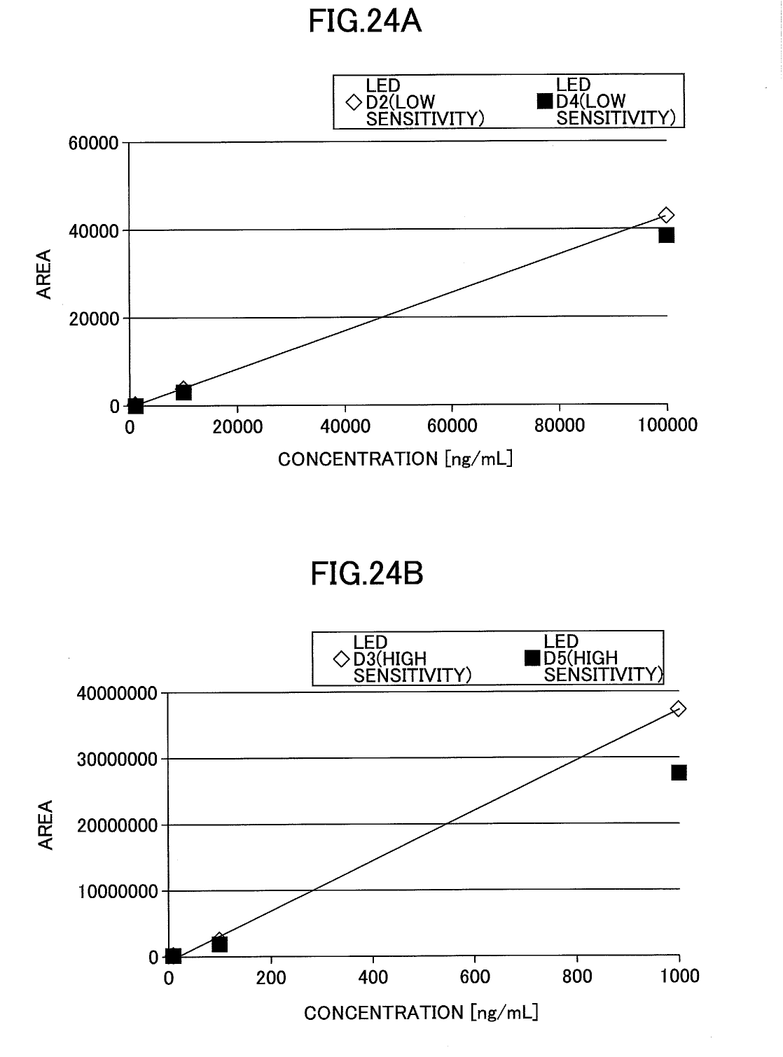

In FIG. 16, the HPLC system 3 includes a fluorescence detection apparatus 100B that is additionally provided downstream of the fluorescence detection apparatus 100A of the HPLC system 2 shown in FIG. 15. When the HPLC system 3 including the fluorescence detection apparatuses 100A and 100B was used in Example 5 described below to analyze a reference standard of fluorescein, which is a fluorescent dye, the analysis results showed good linearity over a range extending 6 orders of magnitude and desired reproducibility between the detection apparatuses (FIGS. 24A and 24B).

By setting one of the two detection apparatuses as a reference apparatus, a performance test can be conducted with respect to the other apparatus. With a simple optical system as described above, a compact structure can be designed by arranging two fluorescence detection apparatuses in close proximity to each other, and the fluorescence detectors can be connected to each other with shorter pipes. In this way, the time it takes for a sample to pass through the pipes and diffusion/chemical change (e.g., decomposition) of the sample while passing through the pipes may be minimized to thereby improve reproducibility between the two fluorescence detection apparatuses.

Note that in some embodiments, the MS apparatus 500 and the computer 400 shown in FIG. 14 may be connected to the HPLC system 2 according to the third embodiment or the HPLC system 3 according to the fourth embodiment.

Although certain preferred embodiments of the present invention have been described above, the present invention is not limited to the above-described embodiments, and various changes and modifications may be made without departing from the scope of the present invention.

### EXAMPLES

In the following, Examples 1 to 4 in which two-dimensional liquid chromatography analysis was performed using a liquid chromatography apparatus will be described.

Note that in Examples 1 to 4, measurements were made using the two-dimensional HPLC system 1 having the flow path configuration as shown in FIG. 1 as the liquid chromatography apparatus. The system was configured according to the following specifications and were operated using the following operating conditions.

<Equipment>

The first pump 21, the second pump 22, the sample injection unit (autosampler) 30, the degassing apparatus 80, the fluorescence detectors 51, 52, and 53, the flow path switching valve 60, and the like were manufactured by Shiseido Co., Ltd.

In the multi-loop unit 70, nine loops 71, each 1500 mm×0.5 mm i.d. (300 μL volume), were connected and used.

Also, columns of different sizes and properties were used as the first dimension reversed phase column 41 and the second dimension chiral column 42 (including chiral columns 42a, 42b, and 42c). The first dimension reversed phase column 41 was arranged to have a size of (0.53 mm i.d.×1000 mm). Each of the second dimension chiral columns 42a, 42b, and 42c was arranged to have a size of (1.5 mm i.d.×250 mm).

<Chiral Amino Acid Analysis>

(1) NBD Derivatization Reaction of Reference Standard Amino Acids

For each amino acid, 10 μL of a 400 mM borate buffer solution (pH 8.0) and 5 μL of a 40 mM NBD-F acetonitrile solution were added to an aqueous solution containing 2.5 μM of the D-form amino acid and 10 μM of the L-form amino acid and the solution was heated at 60° C. for 2 minutes (exceptions to the above amino acid concentrations were 10 μM of glycine (Gly); 12.5 μM of the D-form and 50 μM of the L-form of methionine (Met), lysine (Lys), and cysteine (Cys); 25 μM of the D-form and 100 μM of the L-form of tryptophan (Trp); and 125 μM of the D-form and 500 μM of the L-form of tyrosine (Tyr)).

After adding 75 μL of a 0.1% TFA 30% acetonitrile solution, and diluting the solution ten times with a 0.5% TFA aqueous solution, 20 μL of the resulting sample was analyzed under HPLC conditions (2) described below.

(2) HPLC Conditions

<First Dimension>

Column: maintained at 45° C.

Excitation wavelength: 470 nm; fluorescence wavelength: 530 nm

Mobile Phase A: 5% MeCN, 0.045% TFA in water

Mobile Phase B: 85% MeCN in water

Mobile Phase C: 25% MeCN, 0.05% TFA in water

Mobile phase D: 25% THF, 0.05% TFA in water

Gradient conditions are indicated in Table 1 below.

TABLE 1

| TIME(min) | A % | B % | C % | D % |
|---|---|---|---|---|
| 0-150 | 100 | 0 | 0 | 0 |
| 150-240 | 40 | 0 | 60 | 0 |
| 240-390 | 20 | 0 | 0 | 80 |
| 390-470 | 60 | 40 | 0 | 0 |
| 470-800 | 0 | 100 | 0 | 0 |
| 800-995 | 100 | 0 | 0 | 0 |

<Second Dimension>

Column: 42a, 42b, 42c were used for different purposes as necessary; all were maintained at 25° C.

Excitation wavelength: 470 nm; fluorescence wavelength: 530 nm

Suitable mobile phases vary depending on each amino acid. The following were used.

His (histidine): 0.07% formate in MeOH/MeCN (30/70, v/v), 300 μL/min

Arg (arginine): MeOH/MeCN (60/40, v/v), 150 μL/min

Ser (Serine), Asn (asparagine), Gln (glutamine), Gly (glycine), Thr (threonine): 0.75% formate in MeOH/MeCN (12.5/87.5, v/v), 200 μL/min

Pro (proline), Met (methionine): 1% formate in MeOH/MeCN (12.5/87.5, v/v), 200 μL/min

Ala (alanine): 0.5% formate in MeOH/MeCN (19/81, v/v), 200 μL/min

Val (valine), allo-Ile (alloisoleucine), Ile (isoleucine), Leu (leucine): 0.15% formate in MeOH/MeCN (85/15, v/v), 200 μL/min

Asp (aspartic acid), Glu (glutamic acid): 0.6% formate in MeOH/MeCN (85/15, v/v), 150 μL/min

allo-Thr (allothreonine): 0.4% formate in MeOH/MeCN (5/95, v/v), 200 μL/min

Trp (tryptophan): 0.2% formate in MeOH/MeCN (5/95, v/v), 175 μL/min

Phe (phenylalanine), Lys (lysine), Tyr (tyrosine): 0.4% formate in MeOH/MeCN (60/40, v/v), 200 μL/min

Cys (cysteine): 3% formate in MeOH/MeCN (25/75, v/v), 250 μL/min

Note that in the following, the above amino acids will be referred to by their corresponding abbreviations as appropriate.

<Fluorescence Detection Apparatus Configuration>

Blue LEDs “HLV-14BL-2 W-NR-SP” manufactured by CCS Inc. were used as the light sources 130 and 230. PMT modules “H9306-01” manufactured by Hamamatsu Photonics Co., Ltd. were used as the detectors 51, 52 and 53. Optical band pass filters “FF01-470/27-25” manufactured by Semrock Co., Ltd. having a bandwidth of 22 nm and a center wavelength of 470 nm were used as the excitation light filters 141 and 241. Optical band pass filters “FF01-542/27-25” manufactured by Semrock Co., Ltd. having a bandwidth of 27 nm and a center wavelength of 542 nm were used as the fluorescent filters 142, 143, and 242.

(3) NBD Derivatization Reaction of Sample

Water and MeOH1 were added to the sample and the mixture was stirred, followed by centrifugation to obtain a supernatant. The supernatant was evaporated to dryness under reduced pressure, and a borate buffer (pH 8.0) and a NBD-F acetonitrile solution was added thereto and heated at 60° C. for 2 minutes. The mixture was then diluted with a TFA aqueous solution and analyzed under the HPLC conditions (2) described above.

### Example 1

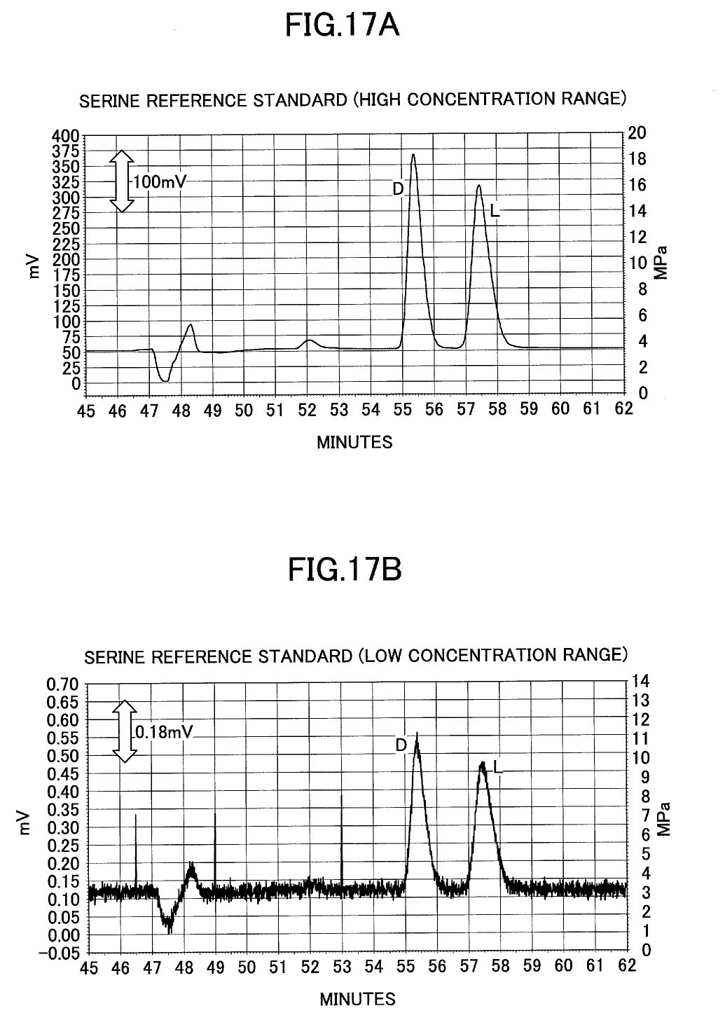

Analysis was performed under the above settings with respect to 500 fmol of NBD-Ser (serine) reference standards with NBD-D/L-Ser (L:D=1:1). FIGS. 17A and 17B show detection results of the second dimension detection obtained by the first detector 52 at 600 V (high concentration range) and the second detector 53 at 1200 V (low concentration range).

### Example 2

To determine the electrical characteristics of detection signals, the time constant and the voltage (photomultiplier voltage) of each detector were examined using the NBD-Ser (serine) reference standards. As a result, it was found that an optimal signal-to-noise ratio (S/N) could be obtained when the time constant was 10 seconds, the photomultiplier voltage of the first dimension detector 51 was 800 V, and the photomultiplier voltages of the first detector 52 and the second detector 53 for the second dimension detection were respectively 600 V (high concentration range) and 1200 V (low concentration range). These values were set up as the optimum values for amino acid detection.

FIG. 18A shows results of conducting a linearity test on Ser (serine) using the fluorescence detection apparatus 100 according to an embodiment of the present invention.

As shown in FIG. 18A, when the NBD-Ser reference standards at nine different concentrations (10 fmol, 50 fmol, 100 fmol, 500 fmol, 1 pmol, 5 pmol, 10 pmol, 50 pmol, 100 pmol) were analyzed under the above conditions, the analysis results showed good linearity over a range extending 6 orders of magnitude.

Upon conducting similar experiments with respect to protein-constituting amino acids in food samples, it was confirmed that the concentration range of the D-forms and L-forms of the protein-constituting amino acids (43 types) could be covered by the quantitation range of one single fluorescence detection apparatus 100. The above results of the linearity test indicate that quantitation with high accuracy can be achieved over a measurement range extending at least 6 orders of magnitude from a detection lower limit of 500 amol to a detection upper limit of 500 pmol.

For example, when 10 fmol was injected, the S/N ratio was 34 (signal (S): 15.5 mV, noise (N): 0.456 mV), and the detection limit (S/N=3) was calculated to be 0.88 fmol.

As a comparison, FIG. 18B shows measurement results of a xenon (Xe) lamp fluorescence detector. The dynamic range of the xenon lamp fluorescence detector under the same conditions extended less than 3 orders of magnitude, the S/N was 6.4 (signal: 1.36 mV; noise: 0.213 mV), and the detection limit was 4.69 fmol.

It can be appreciated from the above comparison that the fluorescence detection apparatus 100 according to the present embodiment can cover a wider measurement range with good linearity. Thus, for example, in the fluorescence detection apparatus according to an embodiment of the present invention, when detecting two different types of analyte components in a sample, even when the amount of one of the analyte components is substantially smaller with respect to amount of the other analyte component in the sample, not only the analyte component with the larger amount but also the analyte component with the smaller amount can be correctly detected and quantitated in one measurement.

### Example 3

The sensitivities of the detections performed by the fluorescence detection apparatus using an LED according to the present embodiment and the fluorescence detection apparatus using a xenon lamp according to the comparative example were compared. FIG. 19A shows detection results obtained by the first detector 52 set to 600 V (high concentration range), and FIG. 19B shows the detection results obtained by the second detector 53 set to 1200 V (low density range).

A prototype of the present specification was able to achieve a quantitation range extending at least 6 orders of magnitude and a sensitivity of at least 1 fmol as shown in FIG. 19A and FIG. 19B, and this was then applied to measure an actual food sample (black vinegar). As a result, D-serine and L-serine could be measured in a manner similar to the case of measuring the reference standards, and the measurement results showed performance (sensitivity/dynamic range) exceeding that of the xenon lamp fluorescence detector.

As can be appreciated from these results, even when the abundance ratios of L-serine and D-serine in black vinegar differ by at least 3 orders of magnitude, by installing two detectors with different detection sensitivities, both L-serine and D-serine can be detected and quantitated.

### Example 4

In the present example, in order to verify the measurement accuracy of the fluorescence detection apparatus of the present embodiment, the mass spectrometer 500 and the computer 400 as shown in FIG. 14 were connected to the liquid chromatography apparatus 1 corresponding to the two-dimensional HPLC system to compare measurements.

In the present example, ACQUITY QDa manufactured by Nihon Waters Co., Ltd. (hereinafter referred to as “MS apparatus”) was used as the mass spectrometer 500.

In the present example, the MS apparatus 500 was tuned using NBD-amino acid reference standards, the sampling rate was set to 1 Hz, the capillary voltage was set to 0.8 kV, the cone voltage was set to 15 V, and measurement was conducted in full scan mode (100.00-400.00 Da). As a result, the NBD-amino acids could only be detected in negative mode.

In turn, measurement results (MS data) obtained by the MS apparatus 500 upon measuring NBD-Ala (251.00), NBD-Leu (293.10), NBD-Asp (295.00) and NBD-Glu (309.00) in SIR (selective ion recording) mode were compared with fluorescence detection data of the same analytes obtained by the fluorescence detection apparatus 100.

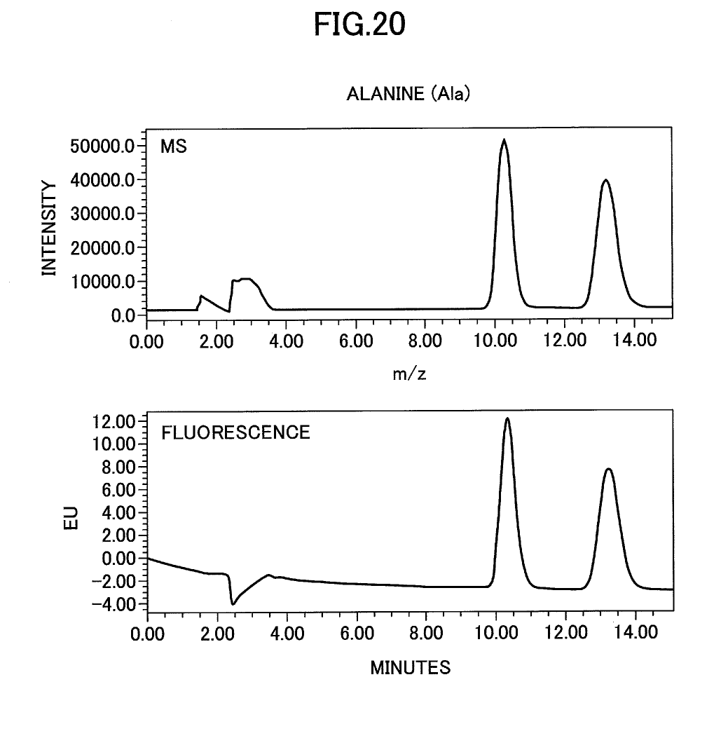

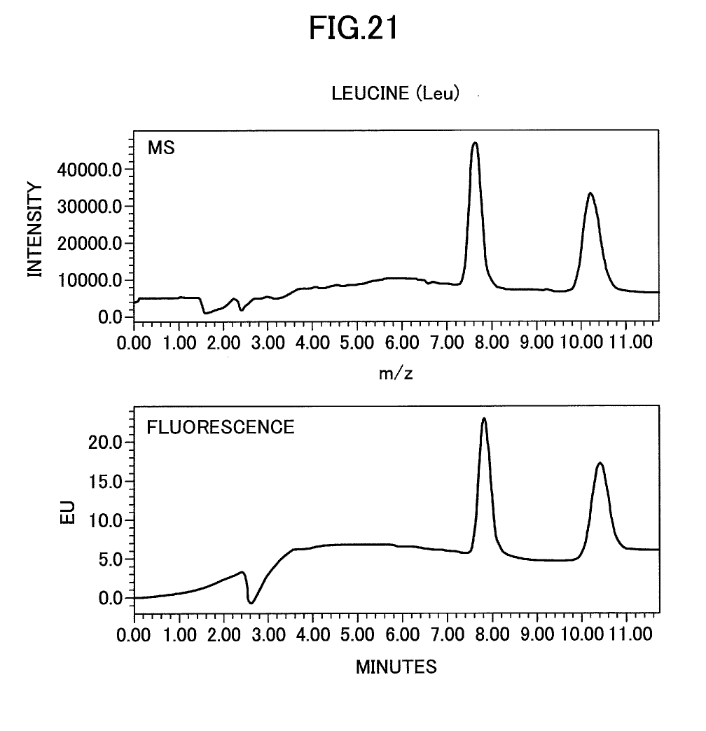

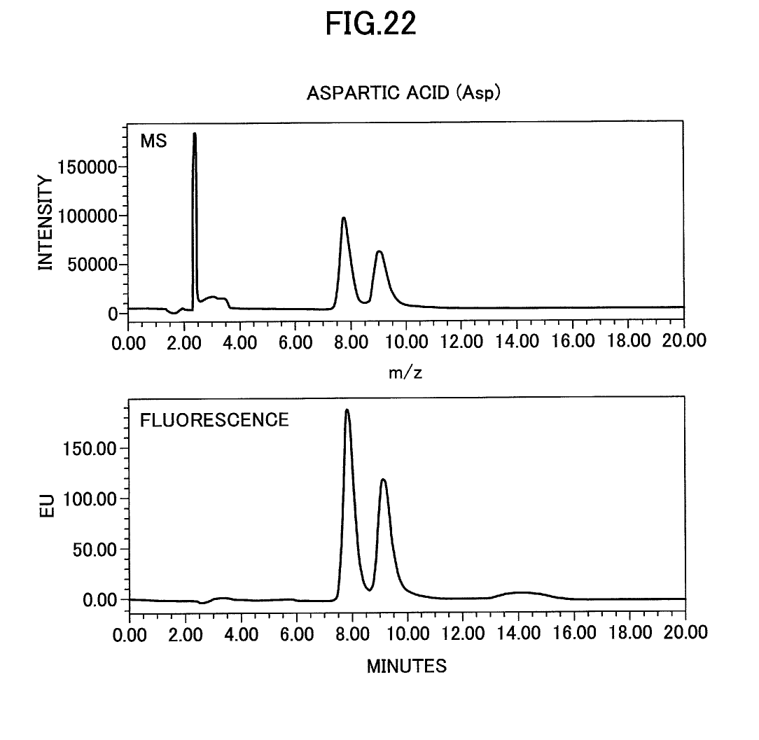

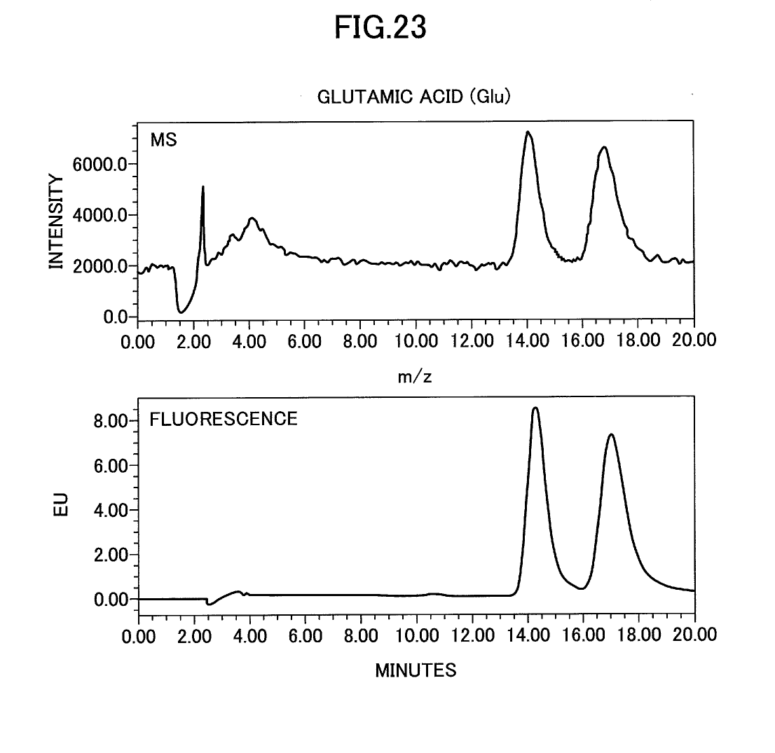

FIG. 20 shows the MS data of Ala and the fluorescence detection data of Ala. FIG. 21 shows the MS data of Leu and the fluorescence detection data of Leu. FIG. 22 shows the MS data of Asp and the fluorescence detection data of Asp. FIG. 23 shows the MS data of Glu and the fluorescence detection data of Glu.

As can be appreciated from FIGS. 20 to 23, the MS data and the fluorescence detection data relating to the peaks of the analytes are substantially the same, and this suggests that the D-amino acid to L-amino acid ratio (D/N) can be reproduced using the fluorescence detection apparatus 100 according to an embodiment of the present invention. That is, by combining the fluorescence detection apparatus 100 and the MS apparatus 500, complementary detection may be performed to further improve detection accuracy.

### Example 5

In the following, Examples 5 and 6 relating to liquid chromatography analyses performed using a liquid chromatography apparatus with the following specifications will be described. In Examples 5 and 6, the HPLC system 3 with the flow path configuration as shown in FIG. 16 was used as the liquid chromatography apparatus, and the system was configured according to the following specifications using the following apparatus conditions and operating conditions.

Column 41: 2 mm×150 mm S-3 MG

Connection Pipes: [Column 41]—[Pipe (diameter φ=0.13 nm; length L=600 mm)—[Cell 110A for D2 and D3 detection]—(Pipe×2 (diameter ϕ=0.13 mm; length L=600 mm)—[Cell 110B for D4 and D5 detection]

Mobile Phase: MeOH/0.05% Formic Acid Water=50/50

Mobile Phase Flow Rate: 200 uL/min

Standard: Fluorescein at 6 different concentrations (1 ng/mL, 10 ng/mL, 100 ng/mL, 1,000 ng/mL, 10,000 ng/mL, 100,000 ng/mL)

Injection Amount: 1 uL

The fluorescence detectors used in the present example were designed so that their detection sensitivities can be set to 6 different levels including H3, H2, H1, MID, L2, L1 (in descending order of detection sensitivity). In the upstream fluorescence detection apparatus 100A, the detector D2 (52A) was set to a low sensitivity (lowest level L1) for covering a high concentration measurement range, and the detector D3 (53A) was set to high sensitivity (the highest level H3) for covering a low concentration measurement range. In the downstream fluorescence detection apparatus 100B, the detector D4 (52B) was set to the low sensitivity L1 for covering the high concentration measurement range and the detector D5 (53B) was set to the high sensitivity H3 for covering the low concentration measurement range.

Also, the sampling rate was set to 5 Hz and 2 Hz, and the time constant (smoothing time) was set to 2 sec (in the case of 5 Hz) and 5 sec (in the case of 2 Hz). Detections under the above settings (5 Hz, 2 sec; and 2 Hz, 5 sec) were respectively performed by the detectors (D2 and D3) and the detectors (D4 and D5) according to the flow of the HPLC system 3 as shown in FIG. 16. Note that detection by the detectors D2 and D3 was performed simultaneously, and detection by the detectors D4 and D5 was performed simultaneously immediately thereafter.

A linearity test was conducted with respect to fluorescein detection results obtained by the HPLC system 3 of the present invention that was set up in the above-described manner.

Further, as a comparative example, fluorescein was detected using a conventional xenon (Xe) lamp fluorescence detection apparatus. The xenon lamp fluorescence detection apparatus was appropriately set up to maximize its measurement range (sensitivity width).

Also, the sampling rate was set to 5 Hz and the time constant (smoothing time) was set to 2 sec as in the LED fluorescence detection apparatuses 100A and 100B. Further, the mobile phases and standards used were the same as those used in the detection by the above HPLC system 3.

Note that because the xenon lamp fluorescence detection apparatus was provided with one detector per unit, separate analyses were conducted using the highest sensitivity and the lowest sensitivity instead of conducting the analyses simultaneously.

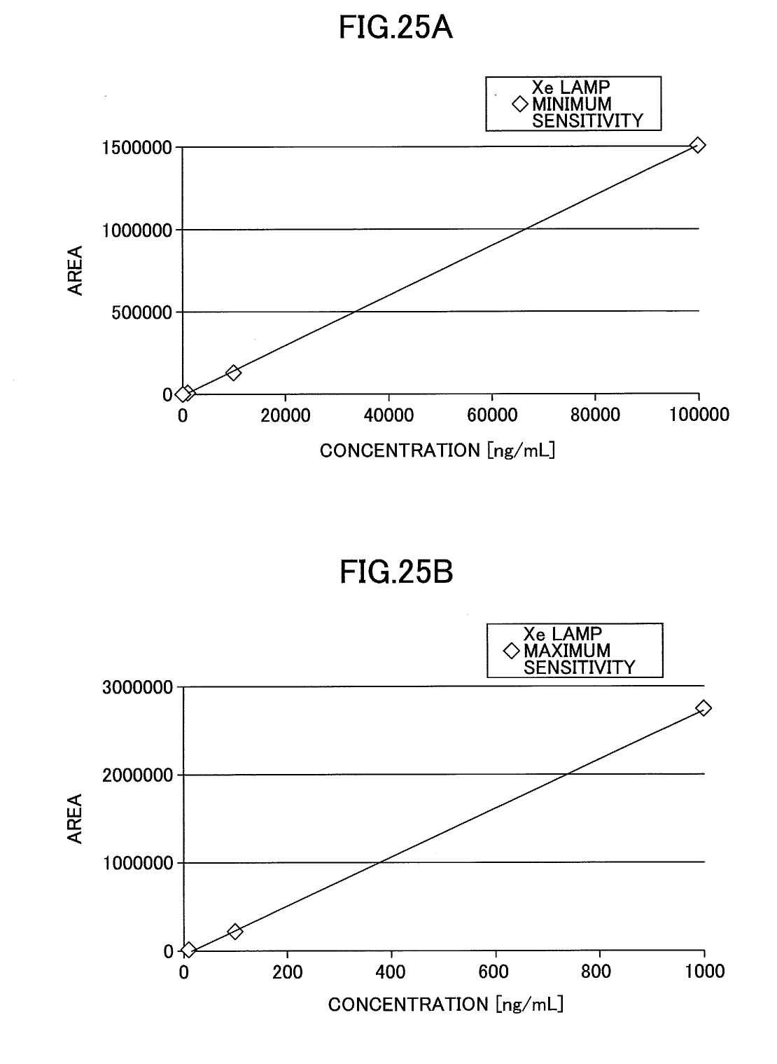

FIGS. 24A and 24B show measurement results of a linearity test conducted with respect to the fluorescein detection results obtained by the HPLC system 3 of FIG. 16. FIGS. 25A and 25B show measurement results of a linearity test conducted with respect to the fluorescein detection results obtained by the xenon lamp fluorescence detection apparatus according to the comparison example. The linearity test results of the fluorescein detection by the xenon lamp fluorescence detection apparatus according to the comparison example and the linearity test results of the fluorescein detection by the HPLC system 3 according to an embodiment of the present invention are shown in Table 2 below.

TABLE 2

| SN | | | | | | | | | | |
|---|---|---|---|---|---|---|---|---|---|---|
| RATIO | | | | | | | | | Xe | |
| CONCEN- | | | | | | | | | MAXIMUM | MINIMUM |
| TRATION | LED-FL 5 Hz Tc = 2 sec | | | | LED-FL Rate = 2 Hz Tc = 5 sec | | | | SENSI- | SENSI- |
| [ng/mL] | ch2(L1) | ch3(H3) | ch4(L1) | ch5(H3) | ch2(L1) | ch3(H3) | ch4(L1) | ch5(H3) | TIVITY | TIVITY |
| 1 | ND | 2.87 | ND | 2.08 | ND | 4.59 | ND | 4.24 | 1.65 | ND |
| 10 | ND | 11 | ND | 8.41 | ND | 17.1 | ND | 12.4 | 9.73 | ND |
| 100 | ND | 81.7 | ND | 76.5 | ND | 162 | ND | 160 | 109 | 7.7 |
| 1,000 | 5.34 | 1512 | 4.18 | 1177 | 5.34 | 2418 | 4.2 | 2190 | 1252 | 93 |
| 10,000 | 83.4 | OVER | 70.8 | OVER | 129 | OVER | 51.3 | OVER | OVER | 1153 |
| 100,000 | 1116 | OVER | 763 | OVER | 1621 | OVER | 766 | OVER | OVER | 10689 |

Referring to FIGS. 25B and 24B, 1 ng/mL to 1,000 ng/mL of fluorescein was detected using the highest sensitivity setting of the xenon lamp fluorescence detection apparatus according to the comparative example (FIG. 25B) and the highest sensitivity setting (H3) of the LED fluorescence detection apparatus according to an embodiment of the present invention (FIG. 24B). The graphs of FIGS. 24B and 25B both show good linearity indicating that adequate measurement ranges for quantitation were obtained.

Also, referring to FIGS. 25A and 24A, 1,000 ng/mL to 100,000 ng/mL of fluorescein was detected using the lowest sensitivity setting of the xenon lamp fluorescence detection apparatus according to the comparative example (FIG. 25A) and the lowest sensitivity setting (L1) of the LED fluorescence detection apparatus 100A according to an embodiment of the present invention (FIG. 24A). The graphs of FIGS. 24A and 25A both show good linearity indicating that adequate measurement ranges for quantitation were obtained.

As can be appreciated from the above, in order to obtain a quantitative measurement range extending over at least 6 orders of magnitude, analysis had to be performed twice using the conventional xenon lamp fluorescence detection apparatus, whereas analysis only had to be performed once when using the LED fluorescence detection apparatus according to an embodiment of the present invention.

### Example 6

Samples of fluorescein at concentrations of 1,000 ng/mL and 10,000 ng/mL were analyzed under the following setting conditions: (1) sampling rate 5 Hz and time constant (smoothing time) 2 sec; and (2) sampling rate 2 Hz and time constant (smoothing time) 5 sec.

FIG. 26A shows detection results of detecting 1,000 ng/mL of fluorescein using the system of FIG. 16 with the sampling rate set to 5 Hz and the time constant set to 2 sec, and FIG. 26B shows detection results of detecting 1,000 ng/mL of fluorescein using the system of FIG. 16 with the sampling rate set to 2 Hz and the time constants set to 5 sec.

Also, FIG. 27A shows detection results of detecting 10,000 ng/mL of fluorescein using the system of FIG. 16 with the sampling rate set to 5 Hz and the time constant set to 2 sec, and FIG. 27B shows detection results of detecting 10,000 ng/mL of fluorescein using the system of FIG. 16 with the sampling rate set to 2 Hz and the time constant set to 5 sec.

In FIG. 26A, FIG. 26B, FIG. 27A, and FIG. 27B, the detectors D2 and D3 perform simultaneous detection, and the detectors D4 and D5 perform simultaneous detection immediately thereafter. It can be appreciated from the above data that sensitivities and dynamic ranges of the same order can be reproduced between the separate fluorescence detection apparatuses, thereby indicating that they have specifications suitable for mass production.

Further, by comparing FIG. 26A with FIG. 26B and comparing FIG. 27A with FIG. 27B, it can be appreciated that setting differences in the sampling rate and the time constant have little impact on the detection sensitivity, and these setting conditions may be suitably set up according to the resolution required for analysis and the signal-to-noise ratio, for example.

Note that although detection was performed for the purpose of detecting fluorescein in the example described above, as shown in FIGS. 27A and 27B, secondary substances (impurities, artifacts) could also be detected by the high sensitivity detection of 10,000 ng/mL of fluorescein.

The ability to observe such a phenomenon could be attributed to the fact that the fluorescence detection apparatus according to an embodiment of the present invention has a substantially wider dynamic range as compared to the conventional fluorescence detection apparatus, and as such, the fluorescence detection apparatus according to present embodiment may be advantageously applied to detection and quantitation of impurities in chemical products and trace components in biological samples, for example.

### DESCRIPTION OF THE REFERENCE NUMERALS

1 Two-dimensional HPLC system (liquid chromatography apparatus)
2, 3 HPLC system (liquid chromatography apparatus)
3 Mass spectrometer (MS apparatus)
11 First mobile phase supply unit
12 Second mobile phase supply unit
21 First pump
22 Second pump
30 Sample injection unit
41 Reversed phase column
42 (42a, 42b, 42c) Chiral column
45 Reversed phase column thermostatic chamber
46 (46a, 46b) Chiral column thermostatic chamber
47a, 47b, 47c Adjustment flow path switching valve
51 First dimension detector
52, 52A, 52B First detector (Second dimension detector)
53, 53A, 53B Second detector (Second dimension detector)
55 Case (for first dimension detector)
56 Case (for first detector)
57 Case (for second detector)
60 Flow path switching valve
70 Multi-loop unit
71 Loop
72 Switching means
80 Degassing apparatus
91 First dimension data processing unit
92 (First recorder) Second dimension data processing unit
93 (Second recorder) Second dimension data processing unit
92A (First recorder) Data processing unit
93A (Second recorder) Data processing unit
98 (First recorder) Downstream data processing unit
99 (Second recorder) Downstream data processing unit
100 Fluorescence detection apparatus (Second dimension fluorescence detection apparatus)
100A Fluorescence detection apparatus
100B Fluorescence detection apparatus (downstream fluorescence detection apparatus)
110, 110A, 110B Cell (Flow cell)
110a Excitation surface (Incident surface)
110b First detection surface
110c Opposing surface
110d Second detection surface
111, 114 Transparent quartz
112, 113 Black quartz
115 Solution passing portion (Sample passing portion)
119 Housing (for cell)
121, 122, 123, 124, 125, 126 Mirror part (Reflection mirror)
130 Light source
139 Housing (for light source)
141 Excitation light filter
142 First fluorescence filter
143 Second fluorescence filter
200 Fluorescence detection apparatus (First dimension fluorescence detection apparatus)
210 Cell
300 Integrated detection apparatus
400 PC (Computer)
500 Mass spectrometer (MS apparatus)

1. A fluorescence detection apparatus comprising:
a cell into which an analyte of a sample is introduced;
a light source configured to irradiate excitation light on the analyte in the cell;
a first detector configured to detect fluorescence generated from the analyte after the excitation light has been irradiated on the analyte; and
a second detector configured to detect the fluorescence generated from the analyte after the excitation light has been irradiated on the analyte;
wherein the second detector detects the fluorescence with a measurement range that is different from a measurement range of the first detector, and
wherein the first detector and the second detector detect the fluorescence simultaneously.

2. The fluorescence detection apparatus according to claim 1, wherein the light source is a light emitting diode.

3. The fluorescence detection apparatus according to claim 2, wherein the first detector and the second detector are arranged on a surface of the cell.

4. The fluorescence detection apparatus according to claim 3, further comprising:
a first fluorescence filter configured to pass a predetermined fluorescence wavelength generated from the analyte, the first fluorescence filter being arranged in contact with the surface of the cell, and the first fluorescence detector being arranged in contact with the first fluorescence filter; and
a second fluorescence filter configured to pass the predetermined fluorescence wavelength, the second fluorescence filter being arranged in contact with the surface of the cell, and the second detector being arranged in contact with the second fluorescence filter.

5. The fluorescence detection apparatus according to claim 4, further comprising:
an excitation light filter that is arranged in contact with an incident surface of the cell, the incident surface being irradiated with the excitation light irradiating the analyte, and the LED as the light source being arranged in contact with the excitation light filter;
wherein the surface of the cell on which the first detector and the second detector are arranged is different from the incident surface; and
wherein the LED, the excitation light filter, the cell, the first fluorescence filter, the first detector, the second fluorescence filter, and the second detector are integrally connected to each other.

6. The fluorescence detection apparatus according to claim 1, wherein
the cell includes a transparent quartz block and a black quartz block;
the transparent quartz block is arranged at a light incidence location of the excitation light irradiated from the light source and a fluorescence emitting location at which fluorescence is emitted to the first detector and the second detector;
the cell includes a central portion that forms a sample passing portion through which the sample including the analyte is passed;
the black quartz block is bonded to each side end of the transparent quartz block that is arranged on an incident surface on which the excitation light is incident; and
a reflection mirror is deposited on an opposing surface opposing the incident surface.

7. The fluorescence detection apparatus according to claim 1, wherein
the first detector is arranged on a first surface that is lateral to an incident surface on which the excitation light is incident; and
the second detector is arranged on a second surface opposing the first surface, the second surface being different from the incident surface.

8. The fluorescence detection apparatus according to claim 1, wherein
the sample includes a first component and a second component, which is included at a lower concentration than the first component;
the first detector performs detection with a first measurement range to detect the first component of the sample; and
the second detector performs detection with a second measurement range that is higher than the first measurement range to detect the second component of the sample.

9. An analysis method comprising steps of:
irradiating excitation light with a predetermined excitation wavelength on a cell into which an analyte of a sample has been introduced; and
simultaneously detecting fluorescence generated from the analyte with a first sensitivity and a second sensitivity, which is different from the first sensitivity, using a first detector and a second detector, the fluorescence detected by the first detector and the second detector being generated from the analyte when the analyte returns to a ground state after the analyte in the cell has been excited by the excitation light irradiated thereon.

10. The analysis method according to claim 9, wherein the sample includes an amino acid or an amino acid derivative including optical isomers.

11. The analysis method according to claim 9, wherein the sample includes fluorescein.

12. A fluorescence detection system comprising:
a sample loading unit configured to load a sample;
a separation unit configured to separate a specific analyte from the sample; and
the fluorescence detection apparatus according to claim 1.

13. The fluorescence detection system according to claim 12, wherein
the analyte in the sample is an amino acid or an amino acid derivative including optical isomers;
the separation unit performs optical resolution of the amino acid or the amino acid derivative by having the fluorescence detection apparatus detect the optical isomers of the amino acid or the amino acid derivative.
